(19) United States
(12) Patent Application Publication
(10) Pub. No.: US 2022/0047608 A1
(43) Pub. Date: Feb. 17, 2022

(54) Method of Treatment and Pharmaceutical Composition for Morning Hypertension

(71) Applicant: Dharma Laboratories LLC, Morrisville, NC (US)

(72) Inventor: Annadurai DHARMARAJAN, Morrisville, NC (US)

(21) Appl. No.: 16/992,956

(22) Filed: Aug. 13, 2020

(51) Int. Cl. A61K 31/5513; A61K 9/00; A61K 31/352; A61K 31/495; A61K 31/4422; A61K 31/40; A61K 31/4365; A61K 31/616; A61P 9/12

(52) U.S. Cl. CPC A61K 31/5513; A61K 9/0053; A61K 31/352; A61K 31/495; A61P 9/12; A61K 31/40; A61K 31/4365; A61K 31/616; A61K 31/4422

(57) ABSTRACT

The disclosure relates to methods and compositions for the treatment of one or more of morning hypertension and nocturnal hypertension.

### FIELD OF THE INVENTION

The present disclosure relates to compositions and methods for treating morning hypertension and nighttime hypertension. In particular, the present disclosure relates to the treatments of disorders where standard hypertensive agents fail to control blood pressure adequately during morning and overnight time frames. The present disclosure includes a novel combination of active ingredients as well as to solid dosage forms of such combinations.

### BACKGROUND OF THE INVENTION

Blood pressure varies throughout the day depending on physical activity, and emotional stress. Unfortunately, for some people, their blood pressure may be too high in the morning. This is called morning high blood pressure or morning hypertension. Likewise, during night, it is called as nocturnal hypertension. Researchers have found that morning and night hypertension increases the risk of heart and blood vessel problems such as stroke. Even in patients with well-controlled blood pressure, 50% still have high morning blood pressure. See, Pharma Times 2007 Apr. 1 05:00:00 Kelli Gibson, PharmD, and Robert Lee Page II, PharmD, FASCP, CGP, BCPS), herein incorporated by reference with regard to such background teaching.

The 2017 American College of Cardiology (ACC) and American Heart Association (AHA) definition of Hypertension (HTN) provides five (5) stages:
a) Normal blood pressure (BP): systolic BP is less than 120, and diastolic BP is less than 80;
b) Elevated BP: systolic BP 120 to 130 and diastolic BP is less than 80;
c) Stage 1 HTN: systolic BP 130 to 139 or diastolic BP 80 to 89;
d) Stage 2 HTN: systolic BP at least 140 or diastolic at least 90; and
e) Hypertensive crises: systolic BP over 180 and/or diastolic BP over 120.

In patients aged 40 to 89 years, each 20-mm Hg increase in systolic BP or 10-mm Hg increase in diastolic BP is associated with a 2-fold increase in mortality from ischemic heart disease and a more than 2-fold increase in mortality from stroke. See, e.g., Lewington S, Clarke R, Qizilbash N, et al. Age-specific relevance of usual blood pressure to vascular mortality: a meta-analysis of individual data for one million adults in 61 prospective studies. Lancet. 2002; 360: 1903-1913, herein incorporated by reference with regard to such background teaching.

The currently available, standard hypertensive medicines do not treat adequately morning or night hypertension for the relevant patient population. There is a need for effective therapeutics which will treat, lower, or reduce high blood pressure during morning and night time hours.

### SUMMARY OF THE INVENTION

One embodiment of the present disclosure includes a method for controlling either morning or nocturnal hypertension to a subject in need thereof comprising administering:

(i) a therapeutically effective amount of one or more of an anxiolytic, sedative, and hypnotic agent;
(ii) a therapeutically effective amount of one or more anti-hypertensive agent.

In one aspect, the anxiolytic, sedative and hypnotic agent is selected from the group consisting of: a benzodiazepine, alprazolam, clonazepam, lorazepam, melatonin, a non-benzodiazepine, zolipderm, zaleplon, eszopiclone, and an orexin receptor antagonist. In one aspect, the anxiolytic, sedative and hypnotic agent is a benzopiazepine. In one aspect, the antihypertensive agent is selected from the group consisting of a diuretic, chlorothizide, furosemide, spironolactone, indapamide, a beta-blockers, atenolol, metoprolol tartrate, bisoprolol fumarate, propanolol hydrochloride, an ACE inhibitor, enalapril, moexipril, Lisinopril, captopril, and angiotensin II receptor blocker, valsartan, telmisartan, irbesartan, a calcium channel blocker, amlodipine besylate, diltiazem hydrochloride, nifedipine, an alpha blocker, doxazosin mesylate, prazosin hydrochloride, terazosin hydrochloride, an alpha-2 receptor agonist, methyldopa, a combined alpha and beta blocker, carvediol, labetalol hydrochloride, a central agonist, clonidine hydrochloride, guanfacine hydrochloride, a peripheral adrenergic inhibitor, guanadrel, reserpine, a vasodilator, minoxidil, and hydralazine hydrochloride. In one aspect, the any-hypertensive is selected from a beta blocker, a calcium channel blocker, a diuretic, or an ACE inhibitor. In one aspect, the agents are administered as a fixed dose combination. In one aspect, the anti-hypertensive is provided as:
a) nebivolol administered in an amount from about 1 mg to about 60 mg;
b) metoprolol administered in an amount from about 10 mg to about 600 mg
c) amlodipine administered in an amount from about 1 mg to about 60 mg;
d) valsartan administered in an amount from about 10 mg to about 650 mg; or
e) hydrochlorothiazide administered in an amount from about 5 mg to about 200 mg.

In one aspect, the sedative is alprazolam administered in an amount of about 0.01 mg to about 10 mg. In one aspect, the therapeutically effective amount of an agent for the present method is less than a standard therapeutic dosage of the agent. In one aspect, the therapeutically effective amount of an agent for the present method is about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90% of the lowest therapeutic dose.

One embodiment of the present disclosure includes a kit comprising:
a) a therapeutically effective amount in unit dose form of one or more of an anxiolytic, sedative, and hypnotic agent;
b) a therapeutically effective amount in unit dose form of one or more anti-hypertensive agent; and
c) instructions for the administration of the agents.

In one aspect, the anxiolytic, sedative and hypnotic agent is selected from the group consisting of: a benzodiazepine, alprazolam, clonazepam, lorazepam, melatonin, a non-benzodiazepine, zolipderm, zaleplon, eszopiclone, and an orexin receptor antagonist. In one aspect, the anxiolytic, sedative and hypnotic agent is a benzodiazepine. In one aspect, the antihypertensive agent is selected from the group consisting of a diuretic, chlorothizide, furosemide, spironolactone, indapamide, a beta-blockers, atenolol, metoprolol tartrate, bisoprolol fumarate, propanolol hydrochloride, an ACE inhibitor, enalapril, moexipril, Lisinopril, captopril, and angiotensin II receptor blocker, valsartan, telmisartan, irbesartan, a calcium channel blocker, amlodipine besylate, diltiazem hydrochloride, nifedipine, an alpha blocker, doxazosin mesylate, prazosin hydrochloride, terazosin hydrochloride, an alpha-2 receptor agonist, methyldopa, a combined alpha and beta blocker, carvediol, labetalol hydrochloride, a central agonist, clonidine hydrochloride, guanfacine hydrochloride, a peripheral adrenergic inhibitor, guanadrel, reserpine, a vasodilator, minoxidil, and hydralazine hydrochloride. In one aspect, the any-hypertensive is selected from a beta blocker, a calcium channel blocker, a diuretic, or an ACE inhibitor. In one aspect, the agents unit dose forms are provided as a single, fixed dose combination. In one aspect, a benzodiazepine and anti-hypertensive agents, wherein the anti-hypertensive is provided as
a) nebivolol in an amount from about 1 mg to about 60 mg;
b) metoprolol in an amount from about 10 mg to about 600 mg
c) amlodipine in an amount from about 1 mg to about 60 mg;
d) valsartan in an amount from about 10 mg to about 650 mg; or
e) hydrochlorothiazide in an amount from about 5 mg to about 200 mg.

In one aspect, the sedative is alprazolam in an amount of about 0.01 mg to about 10 mg. In one aspect, the therapeutically effective amount of an agent for the present method is less than a standard therapeutic dosage of the agent. In one aspect, the therapeutically effective amount of an agent for the present method is about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90% of the lowest therapeutic dose. In one aspect, the kit further comprises a therapeutically effective amount in unit dose form of isosorbide mono- or di-nitrate. In one aspect, the instructions provide guidance on the use of the dose units for:
a. the treatment of a condition or disease selected from the group consisting of: morning hypertension, hypertension, nocturnal hypertension, stroke, peripheral edema, heart failure, congestive heart failure, left ventricular dysfunction, hypertrophic cardiomyopathy, diabetic cardiac myopathy, supraventricular arrhythmia, ventricular arrhythmia, atrial fibrillation, atrial flutter, detrimental vascular remodeling, myocardial infarction, sequelae of myocardial infarction, atherosclerosis, angina, renal insufficiency, angina pectoris, diabetes, secondary aldosteronism, pulmonary hypertension, and renal failure; or
b. the management of a condition or disease selected from the group consisting of: morning hypertension, nocturnal hypertension, peripheral vascular disease, Raynaud's disease, luminal hyperplasia, cognitive dysfunction, glaucoma, and stroke.

One embodiment of the present disclosure includes a pharmaceutical combination composition comprising:
a. a therapeutically effective amount of one or more of an anxiolytic, sedative, and hypnotic agent;
b. a therapeutically effective amount of one or more anti-hypertensive agent; and
c. one or more pharmaceutically acceptable carrier.

In one aspect, each agent individually is provided as an immediate release, or as a delayed, controlled, or extended release formulation. In one aspect, both agents are provided as an immediate release, or as a delayed, controlled, or extended release formulation. In one aspect, the composition is formulated for a subject in need of treating hypertension associated with a definable time period. In one aspect, the time period is morning. In one aspect, the time period is night. In one aspect, the anxiolytic, sedative and hypnotic agent is selected from the group consisting of: a benzodiazepine, alprazolam, clonazepam, lorazepam, melatonin, a non-benzodiazepine, zolipderm, zaleplon, eszopiclone, and an orexin receptor antagonist. In one aspect, the anxiolytic, sedative and hypnotic agent is a benzodiazepine. In one aspect, the antihypertensive agent is selected from the group consisting of a diuretic, chlorothizide, furosemide, spironolactone, indapamide, a beta-blockers, atenolol, metoprolol tartrate, bisoprolol fumarate, propanolol hydrochloride, an ACE inhibitor, enalapril, moexipril, Lisinopril, captopril, and angiotensin II receptor blocker, valsartan, telmisartan, irbesartan, a calcium channel blocker, amlodipine besylate, diltiazem hydrochloride, nifedipine, an alpha blocker, doxazosin mesylate, prazosin hydrochloride, terazosin hydrochloride, an alpha-2 receptor agonist, methyldopa, a combined alpha and beta blocker, carvediol, labetalol hydrochloride, a central agonist, clonidine hydrochloride, guanfacine hydrochloride, a peripheral adrenergic inhibitor, guanadrel, reserpine, a vasodilator, minoxidil, and hydralazine hydrochloride. In one aspect, the hypertensive is selected from a beta blocker, a calcium channel blocker, a diuretic, or an ACE inhibitor. In one aspect, the agents are formulated as a fixed dose combination. In one aspect, the anti-hypertensive is provided as:
a. nebivolol in an amount from about 1 mg to about 60 mg;
b. metoprolol in an amount from about 10 mg to about 600 mg;
c. amlodipine in an amount from about 1 mg to about 60 mg;
d. valsartan in an amount from about 10 mg to about 650 mg; or
e. hydrochlorothiazide in an amount from about 5 mg to about 200 mg.

In one aspect, the sedative is alprazolam in an amount of about 0.01 mg to about 10 mg. In one aspect, the therapeutically effective amount of an agent for the present method is less than a standard therapeutic dosage of the agent. In one aspect, the therapeutically effective amount of an agent for the present method is about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90% of the lowest therapeutic dose. In one aspect, the composition is an oral solid dosage form. In one aspect, the composition is a powder, granule, dripping pill, micro-pellet, tablet, sublingual tablets, sublingual drops, bilayered tablet, matrix tablet, capsule, or lozenge. In one aspect, at least 90% of the benzodiazepine is released in about 30 minutes after administration. In one aspect, at least 90% of the benzodiazepine is released in about 60 minutes after administration. In one aspect, at least 90% of the benzodiazepine is released in about 4 hours after administration. In one aspect, at least 90% of the benzodiazepine is released in about 12 hours after administration. In one aspect, the benzodiazepine is alprazolam. In one aspect, the alprazolam is provided in an amount of about 0.10 mg, about 0.20 mg, about 0.25 mg, about 0.30 mg, about 0.40 mg, about 0.50 mg, about 0.60 mg, about 0.70 mg, about 0.80 mg, about 0.90 mg, about 1.0 mg, about 1.6 mg, about 2.0 mg, about 3.0 mg, or about 5.0 mg. In one aspect, the benzodiazepine is provided in an amount of about 0.025 mg to 40 mg. In one aspect, the benzodiazepine is formulated as a nasal spray, a sublingual spray, an oral solution, or and oral suspension. In one aspect, the one or more pharmaceutically acceptable carrier is selected from the group consisting of: poly methacrylate, poly butyl methacrylate, 2-di methylaminoethyl methacrylate, methyl methacrylate, cellulose esters, carbomers, and combinations thereof. In one aspect, the melatonin is provided in the amount of about 1.0 to about 50 mg.

One embodiment of the present disclosure includes a method of treating either morning or nighttime hypertension for a patient in need thereof comprising administering:
a. a therapeutically effective amount of an agent that affects the sympathetic nervous system; and
b. a therapeutically effective amount of one or more anti-hypertensive agent, wherein the patient's blood pressure is reduced compared to a baseline measurement.

In one aspect, the patient's blood pressure is less than about 135 mm of Hg. In one aspect, the patient's blood pressure is less than about 130 mm of Hg. In one aspect, the patient's blood pressure is less than about 125 mm of Hg. In one aspect, the patient's blood pressure is less than about 120 mm of Hg. In one aspect, the method is for treatment of morning hypertension, the administration of an immediate release formulation is provided within about 1 hour of the subject's waking. In one aspect, the method is for treatment of nighttime hypertension, the administration of an immediate release formulation is provided within about 1 hour of the subject's sleeping. In one aspect, the method is for treatment of morning hypertension, the administration of an extended release formulation is provided within about 8 to about 12 hours of the subject's waking. In one aspect, the method is for treatment of nighttime hypertension, the administration of an extended release formulation is provided within about 8 to about 12 hours of the subject's sleeping.

Some aspects disclosed herein relate to compositions, kits, uses, systems and methods for reducing the risk or treatment of morning hypertension comprising administering a therapeutically effective amount of an oral dosage form with a combination comprising: (i) anxiolytics, sedative—hypnotics, such as benzodiazepines, and (ii) antihypertensive drugs.

One embodiment is a method of treating a subject, comprising: identifying a subject having or at risk of having morning hypertension or nocturnal hypertension; and reducing the risk of stroke or the risk of death from a cardiovascular event by administering to the subject an effective amount of (i) one or more anxiolytic, sedative, and hypnotic drugs, such as alprazolam or a pharmaceutically acceptable salt thereof; and (ii) one or more antihypertensive drugs like nebivolol, or a pharmaceutically acceptable salt thereof effective to reduce the increased risk.

In some aspects the methods are effective to decrease the progression of morning hypertension or nocturnal hypertension in a subject in need thereof. In some embodiments, the decrease in the progression of morning hypertension or nocturnal hypertension is of at least about 5%, 10%, 15%, 20%, 25%, 30%, 35%, 40%, 45%, 50%, 60%, 70%, 80%, or 90% relative to a subject at the same level of progression but who is not receiving treatment by administration of the regimen of the present disclosure.

Numerous clinical studies have shown that lowering blood pressure in hypertensive patients reduces mortality and morbidity. See, Collins R, Peto R, MacMahon S, Hebert P, Fiebach N H, Eberlein K A, Godwin J, Qizilbash N, Taylor J O, Hennekens C H, Lancet 1990, 335(8693):827-38). Despite the availability and use of various classes of agents in the treatment of this medical condition, adequate control of blood pressure is not always achieved (Waeber B, Brunner H R, Am J Hypertens 1997, 10(7 Pt 2):1315-1375), herein incorporated by reference with regard to such background teaching.

In humans, blood pressure (BP) fluctuates according to a circadian rhythm, whereby it decreases during sleep and increases in the morning (Muller et al., 1989, herein incorporated by reference with regard to such background teaching). In line with this circadian rhythm, the onset of adverse cardiovascular and cerebrovascular events such as myocardial infarction, sudden cardiac death, and stroke occur more frequently in the hours of the morning (Willich et al., 1987; Muller et al., 1989; Argentino et al., 1990; Elliot, 1998, each, herein incorporated by reference with regard to such background teaching). The increase in blood pressure observed in the first 2-3 h after waking from nocturnal sleep is known as the morning surge in blood pressure (MSBP; Atkinson et al., 2014, herein incorporated by reference with regard to such background teaching). Importantly, the MSBP has been shown to have prognostic value, as individuals with an accentuated MSBP are more likely to experience deleterious cardiovascular and cerebrovascular events (Kario et al., 2003; Li et al., 2010; Pierdomenico et al., 2014, each, herein incorporated by reference with regard to such background teaching). In addition to an unfavorable prognosis, an accentuated MSBP is associated with left ventricular hypertrophy (Kaneda et al., 2005; Yano et al., 2009, each, herein incorporated by reference with regard to such background teaching.), greater internal carotid intima-media thickness (Yano et al., 2009, herein incorporated by reference with regard to such background teaching), an unfavorable atherosclerotic plaque phenotype (Marfella et al., 2007, herein incorporated by reference with regard to such background teaching) and a prothrombotic environment (Kario et al., 2011, herein incorporated by reference with regard to such background teaching).

Cardiovascular events frequently occur early in the morning, and blood pressure increases from the nighttime to early in the morning due to diurnal changes. As early morning blood pressure is significantly associated with the risk of brain, heart and kidney damage and all cardiovascular risks, morning hypertension, in which the blood pressure is increased in the time of the highest cardiovascular risk, is important. Furthermore, the problem is that the greatest decrease in antihypertensive effect is attenuated early in the morning despite adequate control of clinic blood pressure in hypertensive patients on antihypertensive treatment.

Morning hypertension was considered early morning mean blood pressure ≥130/70 mm Hg, it is associated with two types of circadian blood pressure variation. One is an extension of nocturnal hypertension. The other is the surge type, a rapid increase in blood pressure before and after waking up. Both types of morning hypertension are considered to be possible risk factors for cardiovascular disease.

Nocturnal hypertension (NH) was considered nighttime mean blood pressure ≥120/70 mm Hg, as per current guidelines. From July 2015 to November 2018, 447 patients were analyzed. Mean office blood pressure was 158.6/91.5 mm Hg, and 255 patients (57.0%) were taking at least one antihypertensive drug. Among the 409 (91.5%) valid 24-hour ambulatory blood pressure monitoring (ABPM) 316 (77.3%) showed NH. https://onlinelibrary.wiley.com/doi/full/10.1111/jch.13903

In the cohort of 62,788 treated patients, 52.2% were males and mean age was 61.7±12.8 years. Office SBP/DPB was 150.2±20.1/86.3±12.2 mm Hg and 24-hour ABP was 129.9±14.9/75.2±10.4 mm Hg. Approximately 35% of patients were on monotherapy. Nocturnal hypertension was observed in half of the treated patients (31,277, 49.8%). https://academic.oup.com/ajh/article/27/5/680/2743184

The current disclosure of combination of sedative-hypnotic drugs and antihypertensive drugs treatment targeted at controlling morning hypertension is expected to facilitate complete control of blood pressure over 24 h, including at night, and allows more effective control of cardiovascular events during morning and nocturnal hypertension situations. The target of blood pressure control should be <135/85 mm Hg early in the morning, but control at an even lower level (<130/80 mm Hg over 24 h) is desirable in high-risk hypertensive patients with diabetes or chronic kidney disease.

### BRIEF DESCRIPTION OF THE DRAWINGS

FIG. 1 is a graphic illustrating nighttime and morning hypertension, with reference to Kario, Nocturnal Hypertension, New Technology and Evidence, Hypertension, Vol. 71, No. 6, June 2018, herein incorporated by reference with regarding to the background teaching and definitions of the disorders.

### DETAILED DESCRIPTION OF THE INVENTION

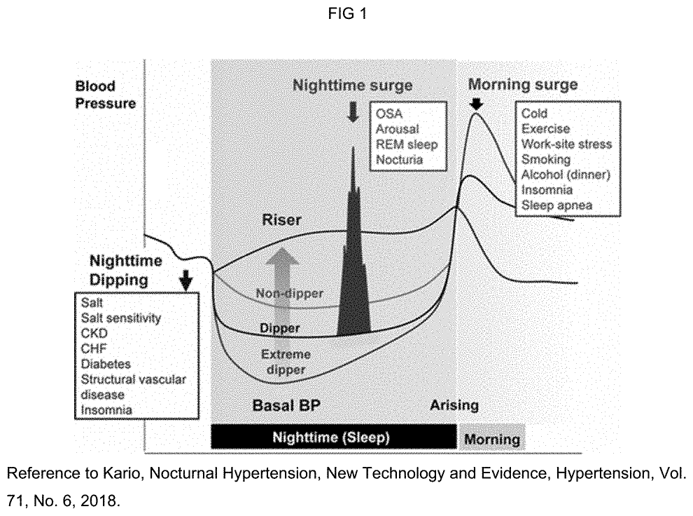

Blood pressure (BP) follows a circadian rhythm, with BP levels falling during sleep and increasing in the early morning hours in most individuals. In patients with hypertension, a lack of decrease in nighttime BP (i.e. non-dipping) and/or a marked rise or surge in BP during the early morning hours is associated with a higher incidence of stroke and an increased risk for other cardiovascular complications, especially among the elderly. Reference is made to FIG. 1.

Morning may be defined as the period between 6 am and 10 am, local time. BP measured in the morning can be used for the diagnosis and therapeutic monitoring of morning hypertension. Morning BP can be assessed by home or ambulatory monitoring or both techniques. For home BP monitoring, morning BP is the average of two or three readings taken within an hour of waking up. If ABPM is performed, morning BP is the average of BP readings within 2 hours of waking up. If the patient's diary on the time of waking up is not available, morning BP would be the average of BP readings in the morning (usually between 6 AM and 10 AM).

Morning hypertension refers to high BP in the morning period, regardless of BP during the rest of the hours of the day. Morning hypertension is defined as morning BP≥135/85 mm Hg for both ABPM and home BP monitoring. Clinic BP measurement can be used for screening morning hypertension. The diagnostic threshold is a BP≥140/90 mm Hg. Morning hypertension in this consensus document includes but is not limited to masked morning hypertension, which was defined in the 2014 Japanese Society of Hypertension Guidelines for the Management of Hypertension as an elevated ABPM or home BP in the morning (≥135/85 mm Hg) and a normal clinic BP (<140/90 mm Hg).

The definition of nocturnal hypertension is nighttime BP≥120/70 mm Hg (>110/65 mm Hg by the 2017 ACC/AHA guidelines). Clinic and morning home BP of <130/80 mm Hg is defined as masked nocturnal hypertension and as masked uncontrolled nocturnal hypertension under a medicated condition. The nighttime BP is calculated as the average of nighttime BPs (from going to bed to arising) measured by ABPM or by the recently developed HBPM system with an automated measurement function for nighttime BP (night-time HBPM). The number of nighttime BP measurements required may be ≥6. The night-time systolic BP dipping (%) is calculated as (1-average night-time systolic BP/average daytime systolic BP)×100, and based on this percentage, the following 4 night-time BP dipping patterns are defined: extreme dipper, >20%; dipper: ≤20%, >10%; nondipper: ≤10%, >0%; riser: ≤0%. This classification is usually based on the ABPM data.

This had led some researchers to recommend the dosing of antihypertensive agents in the evening or bedtime hours in patients with hypertension in order to preserve the normal circadian pattern of BP. See, Lewington S, Clarke R, Qizilbash N, et al. Age-specific relevance of usual blood pressure to vascular mortality: a meta-analysis of individual data for one million adults in 61 prospective studies. Lancet. 2002; 360: 1903-1913, and https://www.ncbi.nlm.nih.gov/books/NBK554579/, each, herein incorporated by reference with regard to such background teaching. An ideal antihypertensive agent would be one that retains its efficacy throughout the 24-h period while also effectively restoring the normal circadian BP pattern. (Dion H. Zappea, Nora Crikelaira, Albert Kandrab, and Paolo Palatinic Journal of Hypertension 2015, 33:385-392, herein incorporated by reference with regard to such background teaching).

Prolonged and uncontrolled hypertensive vascular disease ultimately leads to a variety of pathological changes in target organs such as the heart and kidney. Sustained hypertension can lead as well to an increased occurrence of stroke. The nature of hypertensive vascular diseases is multifactorial. Under certain circumstances, drugs with different mechanisms of action have been combined. However, just considering any combination of drugs having different mode of action does not necessarily lead to combinations with advantageous effects to treat or reduce all kinds of ailments during cardiovascular events.

### Proposed Mechanism of Action for Morning and Nocturnal Hypertension:

The pathophysiology of morning hypertension is not completely understood. Several regulatory mechanisms are thought to or have been found to play a part. The primary cause of the BP increase in the morning may be the activation of the sympathetic nervous system. See, Lambert E A, Chatzivlastou K, Schlaich M, Lambert G, Head G A. Morning surge in blood pressure is associated with reactivity of the sympathetic nervous system. Am J Hypertens. 2014; 27:783-792, herein incorporated by reference with regard to such background teaching. In recent years, with the continuous improvement of living standards and the changes in the dietary structure, the increase of life stress, and the increase of the elderly population is attributing to this morning hypertension and nocturnal hypertension.

The mechanisms contributing to non-dipping or nocturnal hypertension are multifactorial, including hypoxia- and hypercapnea-induced sympathetic nerve activation, renin-angiotensin-aldosterone activation, endothelial dysfunction and increased vascular stiffness. See, Kario K. Obstructive sleep apnea syndrome and hypertension: ambulatory blood pressure. Hypertens Res 2009; 32: 428-432; Kario K, Obstructive sleep apnea syndrome and hypertension: mechanism of the linkage and 24-h blood pressure control. Hypertens Res 2009; 32: 537-541.; Wolf J, Hering D, Narkiewicz K. Non-dipping pattern of hypertension and obstructive sleep apnea syndrome. Hypertens Res 2010; 33: 867-871; and Calhoun D A, Harding S M. Sleep and hypertension. Chest 2010; 138: 434-443, each, herein incorporated by reference with regard to such background teaching.

Target organ damage is more advanced in morning hypertension than in hypertension defined on the basis of clinic blood pressure, and follow-up studies have reported that the risk of stroke a loss of functional independence in those aged 75 years or above is high.

One embodiment of the present disclosure includes a combination comprising components (i) benzodiazepines; and (ii) antihypertensive agents. The components may be administered together, separately and sequentially, in one combined unit dose form or in two or more separate unit dose forms. The unit dose form may also be a fixed combination. A therapeutically effective amount of each of the component of the combination of the present disclosure may be administered simultaneously or sequentially and in any order.

The current disclosure is a combination comprising (i) one or more sedative, hypnotic, or anxiolytic drugs; and (ii) one or more antihypertensive agents, to effectively reduce the incidence of one or more of morning hypertension and nocturnal hypertension. One embodiment of the one or more sedative, hypnotic, or anxiolytic drugs includes benzodiazepines, such as Alprazolam, Clonazepam, or Lorazepam. Other embodiments include melatonin and non-benzodiazepine medicines that calm down the sympathetic nervous system along. Benzodiazepines work by slowing down nerve activity in the brain and the rest of the central nervous system, thereby diffusing stress and its physical and emotional side effects.

The role of the γ-aminobutyric acid (GABA) signaling in blood pressure regulation is complex and only partially characterized. Old studies on anesthetized animals reported that GABA injection in the central nervous system (CNS) decreased blood pressure, heart rate, and renal sympathetic nerve activity. See, Antonaccio M J, Taylor D G, Involvement of central GABA receptors in the regulation of blood pressure and heart rate of anesthetized cats. Eur J Pharmacol 1977; 46:283-287, herein incorporated by reference with regard to such background teaching

Benzodiazepines (BDZ) potentiate the inhibitory effect of GABA in the CNS by binding to GABA_{A}-receptor subunits and increasing the GABA ligand-receptor affinity. Although BDZ are mainly used in the clinic as anxiolytic and hypnotic drugs, they possess also myorelaxant and vasodilatory effects. See, Gielen M C, Lumb M J, Smart T G. Benzodiazepines modulate GABAA receptors by regulating the preactivation step after GABA binding. J Neurosci 2012; 32: 5707-5715; and Klockgether-Radke A P, Pawlowski P, Neumann P, Hellige G. Mechanisms involved in the relaxing effect of midazolam on coronary arteries. Eur J Anaesthesiol 2005; 22:135-139, each, herein incorporated by reference with regard to such background teaching.

Anxiolytics, sedatives and hypnotics are medicines that work on the central nervous system to relieve anxiety, aid sleep, or have a calming effect. The benzodiazepines are one class of drugs that fit into this category. Although there are more than twenty benzodiazepine derivatives, only certain ones have been approved to treat anxiety (e.g., alprazolam, clonazepam, diazepam, and lorazepam), sleeplessness (insomnia) (e.g., estazolam, flurazepam, quazepam, temazepam and triazolam), or panic disorder (e.g., alprazolam). Barbiturates are an older class of medicine that used to be used for these indications as well.

Other drug classes that have a sedative effective include first-generation antihistamines, agonists of melatonin receptors, anesthetics, eszopiclone, zaleplon, zolpidem, zopiclone, and several others. Many of these drugs may also have a hypnotic effect.

The following compounds may be used in the combination of the present disclosure.

### Anxiolytic, Sedative, Hypnotic Component Compound List 1:

Alprazolam (Brand: Xanax tablets 0.25 mg, 0.5 mg, 1 mg, or 2 mg) is a triazolo analog of the 1,4 benzodiazepine class of central nervous system-active compounds. The chemical name of alprazolam is 8-Chloro-1-methyl-6-phenyl-4H-s-triazolo [4,3-α] [1,4] benzodiazepine and alprazolam, as used herein, encompasses a pharmacologically acceptable salt thereof.
Chlordiazepoxide (Brand: Librium capsules 5 mg, 10 mg or 25 mg) is 7-chloro-2-(methylamino)-5-phenyl-3H-1,4-benzodiazepine 4-oxide hydrochloride and chlordiazepoxide, as used herein, encompasses a pharmacologically acceptable salt thereof.
Clonazepam (Brand: Klonopin tablets 0.5 mg, 1 mg and 2 mg) is 5-(2-chlorophenyl)-1,3-dihydro-7-nitro-2H-1,4-benzodiazepin-2-one and clonazepam, as used herein, encompasses a pharmacologically acceptable salt thereof.
Clorazepate (Brand: Tranxene tablets 3.75 mg, 7.5 mg or 15 mg) is a benzodiazepine. The empirical formula is C₁₆H₁₁ClK₂N₂O₄; the molecular weight is 408.92; 1H-1, 4-Benzodiazepine-3-carboxylic acid, 7-chloro-2,3-dihydro-2-oxo-5-phenyl-, potassium salt compound with potassium hydroxide (1:1) and clorazepate cilexetil, as used herein, encompasses a pharmacologically acceptable salt thereof.
Estazolam (Brand: Prosom tablets 1 mg and 2 mg) is a triazolobenzodiazepine derivative. The chemical name for estazolam is 8-chloro-6-phenyl-4H-s-triazolo[4,3-α][1,4]benzodiazepine and estazolam, as used herein, encompasses a pharmacologically acceptable salt thereof.
Flurazepam (Brand: Dalmane tablts 15 mg or 30 mg) is chemically 7-chloro-1-[2-(diethylamino)ethyl]-5-(o-fluorophenyl)-1,3-dihydro-2H-1,4-benzodiazepin-2-one dihydrochloride and flurazepam as used herein encompasses a pharmacologically acceptable salt thereof.
Lorazepam (Brand: Ativan tablets 0.5 mg, 1 mg, or 2 mg) is an antianxiety agent, has the chemical formula, 7-chloro-5-(o-chlorophenyl)-1,3-dihydro-3-hydroxy-2H-1,4-benzodiazepin-2-one and lorazepam as used herein encompasses a pharmacologically acceptable salt thereof.
Midazolam (Brand: Versed tablets 7.5-15 mg) is a short-acting benzodiazepine derivative with an imidazole structure and with anxiolytic, amnestic, hypnotic, anticonvulsant and sedative properties. Midazolam is chemically 8-chloro-6-(2-fluorophenyl)-1-methyl-4H-imidazo[1,5-a][1,4]benzodiazepine and midazolam as used herein encompasses a pharmacologically acceptable salt thereof.
Oxazepam (Brand: Serax capsules 10 mg, 15 mg or 30 mg) is 7-chloro-1,3-dihydro-3-hydroxy-5-phenyl-2H-1,4-benzodiazepin-2-one and oxazepam as used herein encompasses a pharmacologically acceptable salt thereof.
Temazepam (Brand: Restoril capsules 7.5 mg, 15 mg, 22.5 mg, and 30 mg) has the chemical name 7-chloro-1,3-dihydro-3-hydroxy-1-methyl-5-phenyl-2H-1,4-benzodiazepin-2-one and temazepam as used herein encompasses a pharmacologically acceptable salt thereof. Triazolam (Halcion tablets 0.25 mg) is 8-chloro-6-(o-chlorophenyl)-1-methyl-4H-s-triazolo-[4,3-α] [1,4] benzodiazepine and triazolam as used herein encompasses a pharmacologically acceptable salt thereof.
Quazepam (Doral tablets 15 mg) is a trifluoroethyl benzodiazepine hypnotic agent, having the chemical name 7-chloro-5-(o-fluoro-phenyl)-1,3-dihydro-1-(2,2,2-trifluoroethyl)-2H-1,4benzodiazepine-2-thione and quazepam as used herein encompasses a pharmacologically acceptable salt thereof.
Zolpidem tartrate (Brand: Ambien tablets 5 mg and 10 mg) is N,N-6-trimethyl-2-p-tolylimidazo[1,2-α]pyridine-3-acetamide L-(+)-tartrate (2:1) and zolpidem tartrate as used herein encompasses a pharmacologically acceptable salt thereof.
Zolpidem tartrate (Brand: Intermezzo tablets 1.75 mg and 3.5 mg for sublingual administration) is N,N-6-trimethyl-2-p-tolylimidazo[1,2-α]pyridine-3-acetamide L-(+)-tartrate (2:1) and zolpidem tartrate as used herein encompasses a pharmacologically acceptable salt thereof.
Zolpidem tartrate (Brand: Edluar sublingual tablets 5 mg and 10 mg) Chemically, is N,N-6-trimethyl-2-p-tolylimidazo[1,2-α]pyridine-3-acetamide L-(+)-tartrate (2:1) and zolpidem tartrate as used herein encompasses a pharmacologically acceptable salt thereof.
Suvorexant (Brand: Belsomra tablets 5 mg, 10 mg, 15 mg, and 20 mg) is described chemically as: [(7R)-4-(5-chloro-2-benzoxazolyl) hexahydro-7-methyl-1H-1,4-diazepin-1-yl][5-methyl-2-(2H-1,2,3-triazol2-yl)phenyl]methanone and suvorexant as used herein encompasses a pharmacologically acceptable salt thereof.
Butabarbital (Brand: Butisol sodium tablets 30 mg and 50 mg) is chemically described as sodium; 5-butan-2-yl-5-ethyl-4,6-dioxo-1H-pyrimidin-2-olate and butabarbital as used herein encompasses a pharmacologically acceptable salt thereof.
Tasimelteon (Brand: Hetlioz capsules 20 mg) is a melatonin receptor agonist, chemically designated as (1R,2R)—N-[2(2,3-dihydrobenzofuran-4-yl)cyclopropylmethyl]propanamide, containing two chiral centers and tasimelteon as used herein encompasses a pharmacologically acceptable salt thereof.
Eszopiclone (Brand: Lunesta tablets 1 mg, 2 mg and 3 mg) is a nonbenzodiazepine hypnotic agent that is a pyrrolopyrazine derivative of the cyclopyrrolone class. The chemical name of eszopiclone is (+)-(5S)-6-(5-chloropyridin-2-yl)-7-oxo-6,7-dihydro-5H-pyrrolo[3,4-b] pyrazin-5-yl 4-methylpiperazine-1-carboxylate. Eszopiclone has a single chiral center with an (S)-configuration and eszopiclone as used herein encompasses a pharmacologically acceptable salt thereof.
Ramelteon (Brand: Rozerem tablets 8 mg) is an orally active hypnotic chemically designated as (S)—N-[2-(1, 6, 7, 8tetrahydro-2H-indeno-[5, 4-b]furan-8-yl)ethyl]propionamide and containing one chiral center. The compound is produced as the (S)-enantiomer and ramelteon as used herein encompasses a pharmacologically acceptable salt thereof.
Secobarbital (Brand: Seconal sodium capsules 100 mg) is a barbiturate, namely a nonselective central nervous system (CNS) depressants that may be used as a sedative hypnotics. In sub-hypnotic doses, barbiturates are also used as anticonvulsants. Chemically, the drug is sodium 5-allyl-5-(1-methylbutyl) barbiturate and secobarbital as used herein encompasses a pharmacologically acceptable salt thereof.
Doxepin (Brand: Silenor tablets 3 mg and 6 mg) is chemically, doxepin hydrochloride is an (E) and (Z) geometric, isomeric mixture of 1 propanamine, 3-dibenz[b,e]oxepin-11(6H)ylidene-N,N-dimethyl-hydrochloride and doxepin as used herein encompasses a pharmacologically acceptable salt thereof.
and
Zaleplon (Brand: Sonata capsules 5 mg and 10 mg) is a nonbenzodiazepine hypnotic from the pyrazolopyrimidine class. The chemical name of zaleplon is N-[3-(3-cyanopyrazolo[1,5-a]pyrimidin-7-yl)phenyl]-N-ethylacetamide and zaleplon as used herein encompasses a pharmacologically acceptable salt thereof.

There are many classes of antihypertensives, which lower blood pressure by different means. Embodiments of the present disclosure includes thiazide diuretics, calcium channel blockers, ACE inhibitors, angiotensin II receptor antagonists (ARBs), and beta blockers. Many antihypertensive drugs have their primary action on systemic vascular resistance. Some of these drugs produce vasodilation by interfering with sympathetic adrenergic vascular tone (sympatholytics) or by blocking the formation of angiotensin II or its vascular receptors. Other drugs are direct arterial dilators, and some are mixed arterial and venous dilators. Although less commonly used because of a high incidence of side effects, there are drugs that act on regions in the brain that control sympathetic autonomic outflow. By reducing sympathetic efferent activity, centrally acting drugs decrease arterial pressure by decreasing systemic vascular resistance and cardiac output.

Some antihypertensive drugs, most notably beta-blockers, depress heart rate and contractility (this decreases stroke volume) by blocking the influence of sympathetic nerves on the heart. Calcium-channel blockers, especially those (non-dihydropyridines) that are more cardioselective, also reduce cardiac output by decreasing heart rate and contractility. Some calcium-channel blockers (most notably the dihydropyridines) are more selective for the systemic vasculature and therefore reduce systemic vascular resistance.

The following compounds may be used in the combination of the present disclosure.

### Antihypertensive Component Compound List 2A:

### Angiotensin Receptor Blockers (ARBs)

Angiotensin II is a very potent chemical formed in the blood that causes muscles surrounding blood vessels to contract, thereby narrowing the vessels. This narrowing increases the pressure within the vessels and can cause high blood pressure (hypertension). Angiotensin II receptor blockers (ARBs) are medications that block the action of angiotensin II by preventing angiotensin II from binding to angiotensin II receptors on the muscles surrounding blood vessels. As a result, blood vessels enlarge (dilate) and blood pressure is reduced.
Olmesartan medoxomil (Brand: Benicar 10 mg, 20 and 40 mg tablets), its chemical name is (5-methyl-2-oxo-1,3-dioxolan-4-yl)methyl 4-(1-hydroxy-1-methylethyl)-2-propyl-1-[2′-(1H-tetrazol-5-yl)biphenyl-4-y-Imethyl]imidazole-5-carboxylate, and olmesartan medoxomil as used herein encompasses a pharmacologically acceptable salt thereof.
Losartan (Brand: Cozaar, 25 mg, 50 mg and 100 mg tablets) its chemical name is 2-butyl-4-chloro-1-[2′-(1H-tetrazol-5-yl)biphenyl-4-ylmethyl]-1H-imidazol-e-5-methanol, and losartan as used herein encompasses a pharmacologically acceptable salt thereof (losartan potassium salt or the like).
Candesartan cilexetil (Brand: Atacand 4 mg, 8 mg, 16 mg, and 32 mg tablets) its chemical name is 1-(cyclohexyloxycarbonyloxy)ethyl-2-ethoxy-1-[2′-(1H-tetrazol-5-yl)biphen-yl-4-ylmethyl]-1H-benzimidazole-7-carboxylate, and candesartan cilexetil as used herein encompasses a pharmacologically acceptable salt thereof.
Valsartan (Brand: Diovan 40 mg, 80 mg, 160 mg and 320 mg tablets) its chemical name is (S)—N-valeryl-N-[2′-(1H-tetrazol-5-yl)biphenyl-4-ylmethyl) valine, and valsartan as used herein encompasses a pharmacologically acceptable ester thereof or a pharmacologically acceptable salt thereof.
Irbesartan (Brand: Aapro 75 mg, 150 mg, and 300 mg tablets) its chemical name is 2-N-butyl-4-spirocyclopentane-1-[2′-(tetrazol-5-yl)biphenyl-4-ylmethyl]-2-imidazoline-5-one, and irbesartan as used herein encompasses a pharmacologically acceptable salt thereof.
Eprosartan (Brand: Tevetenv 400 mg and 600 mg) its chemical name is 3-[1-(4-carboxyphenyl methyl)-2-n-butyl-imidazol-5-yl]-2-thienyl-methyl-2-propenoic acid, and eprosartan as used herein encompasses a carboxylic acid derivative thereof, a pharmacologically acceptable ester of a carboxylic acid derivative, or a pharmacologically acceptable salt thereof (eprosartan mesylate or the like).
Telmisartan (Brand: Micardis 20 mg, 40 mg or 80 mg tablets) its chemical name is 4′-[[4-methyl-6-(1-methyl-2-benzimidazolyl)-2-propyl-1-benzimidazolyl]met-hyl]-2-biphenylcarboxylic acid, and telmisartan as used herein encompasses a carboxylic acid derivative thereof, a pharmacologically acceptable ester of a carboxylic acid derivative, or a pharmacologically acceptable salt thereof.

### The Calcium Channel Blockers:

Calcium channel blockers decrease the entry of calcium into the cells of the heart and blood vessels. By blocking the entry of calcium, this class of drugs reduces heart rate and contractility and dilates arteries. various agents have been proposed and are actually used in clinical practice, and therefore, those skilled in the art can select a suitable agent exhibiting the effect of the present disclosure
Amlodipine (Brand: Norvasc 2.5 mg, 5 mg, and 10 mg Tablets) is chemically described as 3-Ethyl-5-methyl (±)-2-[(2-aminoethoxy)methyl]-4-(2-chlorophenyl)-1,4-dihydro-6-methyl-3,5-pyridinedicarboxylate, monobenzenesulphonate and amlodipine as used herein encompasses a pharmacologically acceptable salt thereof.
Azelnidipine (Brand: CalBlock 8 and 16 mg tablets) is chemically described 3-[1-(diphenylmethyl)azetidin-3-yl] 5-propan-2-yl 2-amino-6-methyl-4-(3-nitrophenyl)-1,4-dihydropyridine-3,5-dicarboxylate and Azelnidipine as used herein encompasses a pharmacologically acceptable salt thereof.
Aranidipine (Brand: Sapresta) is a novel dihydropyridine derivative that gives rise to two active metabolites (M-1α and M-1β) that exhibit hypotensive activity. It is a calcium antagonist with the formula methyl 2-oxopropyl 1,4-dihydro-2,6-dimethyl-4-(2-nitrophenyl)-3,5-pyridinedicarboxylate. and aranidipine as used herein encompasses a pharmacologically acceptable salt thereof.
Benidipine (Brand: Coniel 4 mg and 8 mg) has the formula 1,4-dihydro-2,6-dimethyl-4-(3-nitrophenyl)-3,5-pyridine-dicarboxylic acid methyl 1-(phenylmethyl)-3-piperidinyl ester hydrochloride. It is a synthetic dihydropyridine derivative and benidipine as used herein encompasses a pharmacologically acceptable salt thereof.
Bepridil (Brand: Vascor 200 mg, 300 mg & 400 mg tablets), the molecular weight of bepridil hydrochloride monohydrate is 421.02. It is chemically described as (±)-(beta)-[(2-Methylpropoxy)methyl]-N-phenyl-N-(phenyl-methyl)-1-yrrolidineethanamine monohydrochloride monohydrate and bepredil as used herein encompasses a pharmacologically acceptable salt thereof.
Cilnidipine (Brand: Atelec 5 mg and 10 mg) is chemically described as 3-(2-methoxyethyl) 5-(2E)-3-phenylprop-2-en-1-yl 2,6-dimethyl-4-(3-nitrophenyl)-1,4-dihydropyridine-3,5-dicarboxylate and cilinidipine as used herein encompasses a pharmacologically acceptable salt thereof.
Clevidipine (Brand: Cleviprex is a sterile IV injection 0.5 ml/ml, 50 mL, 100 mL and 250 mL single use vial) is a dihydropyridine calcium channel blocker. Chemically, the active substance, clevidipine, is butyroxymethyl methyl 4-(2′,3′-dichlorophenyl)-1,4-dihydro-2,6-dimethyl-3,5-pyridinedicarboxylate. It is a racemic mixture with a molecular weight of 456.3 g/mol and clevidipine as used herein encompasses a pharmacologically acceptable salt thereof.
Diltiazem (Brand: Cardizem 30 mg, 60 mg, 90 mg, and 120 mg tablets) is chemically 1,5-Benzothiazepin-4(5H)-one, 3-(acetyloxy)-5-[2-(dimethylamino)ethyl]-2,3-dihydro-2-(4-methoxyphenyl)-, monohydrochloride,(+)-cis- and diazepam as used herein encompasses a pharmacologically acceptable salt thereof.
Felodipine (Brand: Plendil 2.5 mg, 5 mg, and 10 mg tablets) is chemically described as ±ethyl methyl 4-(2,3-dichlorophenyl)1,4-dihydro-2,6-dimethyl-3,5-pyridinedicarboxylate and efonidipine as used herein encompasses a pharmacologically acceptable salt thereof.
Lacidipine (Brand: Lacipil 2, 4 and 6 mg tablets) is chemically described as 3,-5-Pyridinedicarboxilic acid, 4-[2-[3-(1,1-dimetyletoxy)-3-oxo-1-propenyl]phenyl]-1,4-dihydro-2,6-dimetykdietyl ester, (E)- and lacidipine as used herein encompasses a pharmacologically acceptable salt thereof.
Lercanidipine (Brand: Zanidip 10 mg and 20 mg) is a dihydropyridine derivative. It is a racemate due to the presence of a chiral carbon atom at position 4 of the 1,4-dihydropyridine ring. 3,5-pyridinedicarboxylic acid, 1,4-dihydro-2, 6-dimethyl-4-(3-nitrophenyl)-2-[(3,3-diphenylpropyl)methylamino]-1,1-dimethylethyl methyl ester hydrochloride and lercanidipine as used herein encompasses a pharmacologically acceptable salt thereof.
Levamlodipine (Brand: Conjupri 1.25 mg, 2.5 mg, and 5 mg) is chemically described as (S)3-ethyl-5-methyl-2-(2aminoethoxymethyl)-4-(2-chlorophenyl)-1,4-dihydro-6-methyl-3,5-pyridinedicarboxylate maleate and levamlodipine as used herein encompasses a pharmacologically acceptable salt thereof.
Isradipine (Brand: Dynacirc 2.5 mg and 5 mg Capsules) is 3,5-Pyridinedicarboxylic acid, 4-(4-benzofurazanyl)-1,4-dihydro-2,6-dimethyl-, methyl 1-methylethyl ester and isradipine used herein encompasses a pharmacologically acceptable salt thereof.
Manidipine (Brand: Manyper 10 mg and 20 mg) is chemically described as 3-{2-[4-(diphenylmethyl)piperazin-1-yl]ethyl}5-methyl 2,6-dimethyl-4-(3-nitrophenyl)-1,4-dihydropyridine-3,5-dicarboxylate and manidipine as used herein encompasses a pharmacologically acceptable salt thereof.
Nicardipine (Brand: Cardene SR is a sustained release formulation as capsules for oral administration each contain 30 mg, 45 mg or 60 mg) is a dihydropyridine derivative with the IUPAC (International Union of Pure and Applied Chemistry) chemical name (±)-2-(benzyl-methyl amino) ethyl methyl 1,4-dihydro-2,6 dimethyl-4-(m-nitrophenyl)-3, 5-pyridinedicarboxylate monohydrochloride and nicardipine as used herein encompasses a pharmacologically acceptable salt thereof.
Nitrendipine (Brand: Baypress tablets 10 mg and 20 mg) is chemically described as 3-ethyl 5-methyl 2,6-dimethyl-4-(3-nitrophenyl)-1,4-dihydropyridine-3,5-dicarboxylate and nitrendipine as used herein encompasses a pharmacologically acceptable salt thereof.
Nifedipine (Brand: Procardia 10 mg Capsules) is 3,5-pyridinedicarboxylic acid, 1,4-dihydro-2,6-dimethyl-4-(2-nitrophenyl)-dimethyl ester and nifedipine as used herein encompasses a pharmacologically acceptable salt thereof.
Nimodipine (Brand: Nimotop 30 mg liquid filled capsules) is isopropyl 2-methoxyethyl 1, 4-dihydro-2, 6-dimethyl-4-(m-nitrophenyl)-3, 5-pyridinedicarboxylate and nimodipine as used herein encompasses a pharmacologically acceptable salt thereof.
Nisoldipine (Brand: Sular tablets 8.5 mg, 17 mg, and 34 mg) is 3,5-pyridinedicarboxylic acid, 1,4-dihydro-2,6-dimethyl-4-(2-nitrophenyl)-, methyl 2-methylpropyl ester and nisoldipine as used herein encompasses a pharmacologically acceptable salt thereof.
Niguldipine is chemically described as 3-[3-(4,4-diphenylpiperidin-1-yl)propyl] 5-methyl (4S)-2,6-dimethyl-4-(3-nitrophenyl)-1,4-dihydropyridine-3,5-dicarboxylate and niguldipine as used herein encompasses a pharmacologically acceptable salt thereof.
Verapamil (Brand: Verelan 120 mg, 180 mg, 240 mg and 360 mg capsules) is chemically Benzeneacetonitrile, α-[3-[[2-(3,4-dimethoxyphenyl)-ethyl]methylamino]propyl]-3,4-dimethoxy-α-(1-methylethyl), monohydrochloride and verapamil as used herein encompasses a pharmacologically acceptable salt thereof.

A Calcium Channel Blocker (CCB) useful in said combination is preferably selected from the group consisting of amlodipine, diltiazem, felodipine, fendiline, flunarizine, gallopamil, isradipine, lacidipine, mibefradil, nicardipine, nifedipine, niguldipine, niludipine, nimodipine, nisoldipine, nitrendipine, nivaldipine, ryosidine, tiapamil and verapamil, and in each case, a pharmaceutically acceptable salt thereof. All these drugs are therapeutically used as CCBs, e.g. as anti-hypertensive, anti-angina pectoris or anti-arrhythmic drugs.

Preferred CCBs comprise amlodipine, diltiazem, isradipine, nicardipine, nifedipine, nimodipine, nisoldipine, nitrendipine, and verapamil, or, e.g. dependent on the specific CCB, a pharmaceutically acceptable salt thereof. Especially preferred is amlodipine or a pharmaceutically acceptable salt, especially the besylate, thereof.

The compounds to be combined can be present as pharmaceutically acceptable salts. If these compounds have, for example, at least one basic center, they can form acid addition salts. Corresponding acid addition salts can also be formed having, if desired, an additionally present basic centre. The compounds having at least one acid group (for example COOH) can also form salts with bases. Corresponding internal salts may furthermore be formed, if a compound of formula comprises e.g. both a carboxy and an amino group.

Preferred salts of corresponding CCBs are amlodipine besylate, diltiazem hydrochloride, fendiline hydrochloride, flunarizine di-hydrochloride, gallopamil hydrochloride, mibefradil di-hydrochloride, nicardipine hydrochloride, and verapamil hydrochloride.

The following compounds may be used in the combination of the present disclosure.

### Antihypertensive Component Compound List 2B:

ACE (Angiotensin Converting Enzyme) Inhibitors: ACE inhibitors remain the initial treatment of choice for hypertension. This class of drugs blocks the conversion of angiotensin I to angiotensin II by inhibiting angiotensin-converting enzyme (ACE), thus preventing constriction of blood vessels.
Benezapril (Brand: Lotensin 10 mg, 20 mg, and 40 mg tablets) is 3-[[1-(ethoxy-carbonyl)-3phenyl-(1S)-propyl] amino]-2,3,4,5-tetrahydro-2-oxo-1H-1-(3S)-benzazepine-1-acetic acid monohydrochloride and benezapril as used herein encompasses a pharmacologically acceptable salt thereof.
Captopril (Brand: Capoten 12.5 mg, 25 mg, 50 mg, and 100 mg tablets) is designated chemically as 1-[(2S)-3-mercapto-2-methylpropionyl]-L-proline [MW 217.29] and captopril as used herein encompasses a pharmacologically acceptable salt thereof.
Enalapril (Brand: Vasotec 2.5 mg, 5 mg, 10 mg, and 20 mg tablets) is chemically described as (S)-1[N-[1-(ethoxycarbonyl)-3-phenylpropyl]-L-alanyl]-L-proline, (Z)-2-butenedioate salt (1:1) and enalapril as used herein encompasses a pharmacologically acceptable salt thereof.
Fosinopril (Brand: Monopril 10 mg, 20 mg, and 40 mg tablets) is designated chemically as: L-proline, 4-cyclohexyl-1-[[[2-methyl-1-(1-oxopropoxy)propoxy](4-phenylbutyl)phosphinyl]acetyl]-, sodium salt, trans-. and fosinopril as used herein encompasses a pharmacologically acceptable salt thereof.
Lisinopril (Brand: Prinivil 5 mg, 10 mg, and 20 mg tablets) is chemically described as (S)-1-[N2-(1-carboxy-3-phenylpropyl)-Llysyl]-L-proline dihydrate and lisinopril as used herein encompasses a pharmacologically acceptable salt thereof.
Moexipril (Brand: Univasc 7.5 mg and 15 mg tablets) is chemically described as [3S[2 [R*(R*)],3R*]]-2-[2-[[1-(ethoxycarbonyl)-3-phenylpropyl]amino]-1-oxopropyl]-1,2,3,4tetrahydro-6,7-dimethoxy-3-isoquinolinecarboxylic acid, monohydrochloride and moexipril as used herein encompasses a pharmacologically acceptable salt thereof.
Perindopril (Brand: Aceon 2 mg, 4 mg, and 8 mg tablets) is chemically described as (2S,3DS,7DS)-1-[(S)—N—[(S)-1-Carboxy-butyl]alanyl]hexahydro-2-indolinecarboxylic acid, 1-ethyl ester, compound with tert-butylamine (1:1) and perindopril as used herein encompasses a pharmacologically acceptable salt thereof.
Quinapril (Brand: Accupril 5 mg, 10 mg, 20 mg, and 40 mg tablets) is chemically described as [3S-[2[R*(R*)], 3R*]]-2-[2-[[1(ethoxycarbonyl)-3-phenylpropyl]amino]-1-oxopropyl]-1,2,3,4-tetrahydro-3isoquinolinecarboxylic acid, monohydrochloride and quninapril as used herein encompasses a pharmacologically acceptable salt thereof.
Ramipril (Brand: Altace 1.25 mg, 2.5 mg, 5 mg, and 10 mg capsules) has the chemical name (2S,3aS,6aS)-1[(S)—N—[(S)-1-Carboxy-3phenylpropyl] alanyl] octahydrocyclopenta [b]pyrrole-2-carboxylic acid, 1-ethyl ester and ramipril as used herein encompasses a pharmacologically acceptable salt thereof.
Trandolapril (Brand: Mavik 1 mg, 2 mg, or 4 mg tablets) is chemically described as (2S, 3aR, 7aS)-1-[(S)—N—[(S)-1Carboxy-3-phenylpropyl]alanyl] hexahydro-2-indolinecarboxylic acid, 1-ethyl ester and trandolapril as used herein encompasses a pharmacologically acceptable salt thereof.
Beta-blockers: Beta-blockers produce antihypertensive action by reducing heart rate and cardiac output. Currently beta-blockers are not recommended as first-line treatment due to the risk of stroke and new-onset of type 2 diabetes when compared to other medications.
Beta-Blockers with Alpha Activity
Carvedilol (Brand: Coreg, Coreg CR 3.125 mg, 6.25 mg, 12.5 mg, or 25 mg tablets) is chemically described as (±)-1(Carbazol-4-yloxy)-3-[[2-(o-methoxyphenoxy)ethyl]amino]-2-propanol and carvedilol as used herein encompasses a pharmacologically acceptable salt thereof.
Labetalol (Brand: Trandate 100 mg, 200 mg, and 300 mg tablets) is chemically designated as 2-hydroxy-5-[1-hydroxy-2-[(1methyl-3-phenylpropyl)amino]ethyl]benzamide monohydrochloride and labetalol as used herein encompasses a pharmacologically acceptable salt thereof.

Beta-Blockers with Intrinsic Sympathomimetic Activity

Acebutolol (Brand: Sectral 200 mg and 400 mg capsules) is chemically defined as the hydrochloride salt of (±)N-[3-Acetyl-4-[2-hydroxy-3-[(1-methylethyl)amino]propoxy]phenyl]butanamide and acebutolol as used herein encompasses a pharmacologically acceptable salt thereof.
Pindolol (Brand: Visken 5 mg and 10 mg tablets) is chemically described as 1-(Indol-4-yloxy)-3-(isopropylamino)-2-propanol and pindolol as used herein encompasses a pharmacologically acceptable salt thereof.
Penbutolol (Brand: Levatol 20 mg tablets) is (S)—I-tert-butylamino-3-(o-cyclopentylphenoxy)-2-propanol sulfate and penbutolol as used herein encompasses a pharmacologically acceptable salt thereof.

### Beta-1 Cardio-Selective Beta-Blockers

Atenolol (Brand: Tenormin 25 mg, 50 mg, and 100 mg tablets) is chemically described as benzeneacetamide, 4-[2′-hydroxy-3′-[(1-methylethyl) amino] propoxy]- and atenolol as used herein encompasses a pharmacologically acceptable salt thereof.
Betaxolol (Brand: Kerlone 10 mg and 20 mg tablets) is chemically described as 2-propanol, 1-[4-[2-(cyclopropylmethoxy) ethyl] phenoxy]-3-[(1-methylethyl) amino]-, hydrochloride, (±) and betaxolol as used herein encompasses a pharmacologically acceptable salt thereof.
Bisoprolol (Brand: Zebeta 5 mg and 10 mg tablets) is (±)-1-[4-[[2-(1-Methylethoxy)ethoxy]methyl]phenoxy]-3-[(1-methylethyl)amino]-2-propanol(E)-2-butenedioate (2:1) (salt) and bisoprolol as used herein encompasses a pharmacologically acceptable salt thereof.
Celiprolol (Brand: Selectol) is 1-{3-acetyl-4-[3-(tert-butylamino)-2-hydroxypropoxy]phenyl}-3,3-diethylurea and celiprolol as used herein encompasses a pharmacologically acceptable salt thereof.
Metoprolol (Brand: Lopressor, Toprol XL 25 mg, 50 mg, 100 mg, and 200 mg tablets) is (±)1-(isopropylamino)-3-[p-(2-methoxyethyl) phenoxy]-2-propanol succinate (2:1) (salt) and metoprolol as used herein encompasses a pharmacologically acceptable salt thereof.
Nebivolol (Brand: Bystolic 2.5 mg, 5 mg, 10 mg, and 20 mg tablets) is (1RS,1′RS)-1,1′-[(2RS,2′SR)bis(6-fluoro-3,4-dihydro-2H-1-benzopyran-2-yl)]-2,2′-iminodiethanol hydrochloride and nebivolol as used herein encompasses a pharmacologically acceptable salt thereof.
Sotalol (Brand: Betapace, Betapace AF, Sorine 80 mg, 120 mg, and 160 mg tablets) is d,l-N-[4-[1-hydroxy-2-[(1-methylethyl) amino]ethyl]phenyl]methane-sulfonamide monohydrochloride and sotalol as used herein encompasses a pharmacologically acceptable salt thereof.

### Nonselective Beta-Blockers

Nadolol (Brand: Corgard 20 mg, 40 mg and 80 mg tablets) is designated chemically as 1-(tert-butylamino)-3-[(5,6,7,8-tetrahydro-cis-6,7-dihydroxy-1-naphthyl)oxy]-2-propanol and nadolol as used herein encompasses a pharmacologically acceptable salt thereof.
Propranolol (Brand: Inderal LA, InnoPran XL 60 mg, 80 mg, 120 mg, and 160 mg capsules) is chemically described as 2-Propanol, 1-[(1-methylethyl)amino]-3-(1-naphthalenyloxy)-, hydrochloride,(±)- and propranolol as used herein encompasses a pharmacologically acceptable salt thereof.
Timolol (Brand: Blocadren 5 mg, 10 mg, and 20 mg tablets) is (S)-1-[(1,1-dimethylethyl)amino]-3-[[4-(4-morpholinyl)-1,2,5-thiadiazol-3-yl]oxy]-2-propanol (Z)-2-butenedioate (1:1) salt and timolol as used herein encompasses a pharmacologically acceptable salt thereof.

In some aspects, the beta-blocker is acebutolol, atenolol, betaxolol, bisoprolol, carteolol, esmolol, penbutolol, metoprolol, nadolol, nebivolol, pindolol, sotalol, propranolol, carvedilol, labetalol, timolol, esmolol, celiprolol, oxprenolol, levobunolol, practolol, metipranolol, landiolol, bopindolol, pronethalol, butaxamine, bevantolol, tertatolol, arotinolol, levobetaxolol, befunolol, amosulalol, tilisolol, or the pharmaceutically acceptable salt or hydrate thereof.

In some aspects, the beta-blocker is nebivolol, and the dose of nebivolol is about 2 mg to 50 mg.

The following compounds may be used in the combination of the present disclosure.

Antihypertensive Component Compound List 2C:

Diuretics, Loop: Loop diuretics lower blood pressure by reducing blood volume. These medications inhibit the sodium-potassium-chloride cotransporter in the thick ascending limb of the loop of Henle. Loop diuretics promote water loss and increase sodium excretion.
Bumetanide (Brand: Bumex 0.5 mg, 1 mg, and 2 mg tablets) is 3-(butylamino)-4-phenoxy-5-sulfamoylbenzoic acid and beuetamide as used herein encompasses a pharmacologically acceptable salt thereof.
Ethacrynic acid (Brand: Edecrin 25 mg tablets) is designated chemically as [2,3-dichloro-4-(2-methylene-1-oxobutyl)phenoxy] acetic acid and ethacrynic as used herein encompasses a pharmacologically acceptable salt thereof.
Furosemide (Brand: Lasix 20 mg, 40 mg, and 80 mg tablets) is chemically defined as 4-chloro-N-furfuryl-5-sulfamoylanthranilic acid and furosemide as used herein encompasses a pharmacologically acceptable salt thereof.
Piretanide (Brand: Arelix, Eurelix, Tauliz) is 4-phenoxy-3-(pyrrolidin-1-yl)-5-sulfamoylbenzoic acid and piretanide as used herein encompasses a pharmacologically acceptable salt thereof.
Torsemide (Brand: Demadex 5 mg, 10 mg, 20 mg, and 100 mg tablets) is 1-isopropyl-3 [(4-m-toluidino-3-pyridyl) sulfonyl] urea and torsemide as used herein encompasses a pharmacologically acceptable salt thereof.
Diuretics, Potassium-Sparing: Potassium-sparing diuretics work by leaving more potassium in the blood, as a result more sodium and water are excreted in the urine. Potassium-sparing diuretics are weak antihypertensives when used alone.
Amiloride (Brand: Midamore 5 mg tablets) is designated chemically as 3,5-diamino-6-chloro-N-(diaminomethylene) pyrazinecarboxamide monohydrochloride, dihydrate and amiloride as used herein encompasses a pharmacologically acceptable salt thereof.
Spironolactone (Brand: Aldactone 25 mg, 50 mg, and 100 mg tablets) is designated chemically as 17-hydroxy-7α-mercapto-3-oxo-17α-pregn-4-ene-21-carboxylic acid γ-lactone acetate and spironolactone as used herein encompasses a pharmacologically acceptable salt thereof.
Triamterene (Brand: Dyrenium 50 and 100 mg capsules) is 2,4,7-triamino-6-phenyl-pteridine and triamterene as used herein encompasses a pharmacologically acceptable salt thereof.
Diuretics, Thiazide: Thiazide diuretics reduce sodium absorption from the distal tubule segment of the kidney. Thiazide diuretics are known to worsen insulin sensitivity and elevate serum total cholesterol levels.
Bendroflumethiazide (Brand: Aprinox 5 mg tablets) is 3-benzyl-1,1-dioxo-6-(trifluoromethyl)-3,4-dihydro-2H-1λ⁶,2,4-benzothiadiazine-7-sulfonamide and bendroflumethiazide as used herein encompasses a pharmacologically acceptable salt thereof.
Chlorothiazide (Brand: Diuril 250 mg tablets) is 6-chloro-2H-1,2,4-benzothiadiazine7-sulfonamide 1,1-dioxide and chlorothiazide as used herein encompasses a pharmacologically acceptable salt thereof.
Chlorthalidone (Brand: Hygroton 25 mg and 50 mg tablets) is 2-chloro-5-(1-hydroxy-3-oxo-2,3-dihydro-1H-isoindol-1-yl)benzene-1-sulfonamide and chlorthalidone as used herein encompasses a pharmacologically acceptable salt thereof.
Indapamide (Brand: Lozol 1.25 mg tablets) is 1-(4-chloro-3sulfamoylbenzamido)-2-methylindoline and indapamide as used herein encompasses a pharmacologically acceptable salt thereof.
Hydrochlorothiazide (Brand: Hydrodiuril) is 6-chloro-1,1-dioxo-3,4-dihydro-2H-1λ⁶,2,4-benzothiadiazine-7-sulfonamide and hydrochlorothiazide as used herein encompasses a pharmacologically acceptable salt thereof.
Methyclothiazide (Brand: Enduron 5 mg tablets) is 6-chloro-3-(chloromethyl)-3,4-dihydro-2-methyl-2H-1,2,4-benzothiadiazine-7-sulfonamide 1,1-dioxide and methylclothiazide as used herein encompasses a pharmacologically acceptable salt thereof.
Metolazone (Brand: Zaroxolyn, Diulo, Mykrox 2.5 and 5 mg tablets) is 7-chloro-1, 2, 3, 4-tetrahydro-2-methyl-3-(2-methylphenyl)-4-oxo-6-quinazolinesulfonamide and metolazone as used herein encompasses a pharmacologically acceptable salt thereof.
Peripheral Adrenergic Inhibitors: These medications are rarely used unless other medications prove ineffective.
Guanadrel (Brand: Hylorel) is 2-{1,4-dioxaspiro[4.5]decan-2-ylmethyl}guanidine and guanadrel as used herein encompasses a pharmacologically acceptable salt thereof.
Guanethidine (Brand: Ismelin 10 mg and 25 mg tablets) is [2-(hexahydro-1(2H)-azocinyl)ethyl]guanidine sulfate 1:1 and guanethidine as used herein encompasses a pharmacologically acceptable salt thereof.
Reserpine (Brand: Serpasil 0.1 mg and 0.25 mg tablets) is methyl (1R,15S,17R,18R,19S,20S)-6,18-dimethoxy-17-(3,4,5-trimethoxybenzoyloxy)-3,13-diazapentacyclo[11.8.0.0\hat{ }{2,10}.0\hat{ }{4,9}.0\hat{ }{15,20}]henicosa-2(10),4(9),5,7-tetraene-19-carboxylate and reserpine as used herein encompasses a pharmacologically acceptable salt thereof.
Renin Inhibitors: Renin inhibitors act by inhibiting the activity of renin, the enzyme responsible for angiotensin II levels.
Aliskiren (Brand: Tekturna 150 mg and 300 mg tablets) is chemically described as (2S,4S,5S,7S)—N-(2-carbamoyl-2-methylpropyl)-5-amino-4-hydroxy-2,7-diisopropyl-8-[4-methoxy-3-(3methoxypropoxy)phenyl]-octanamide hemifumarate and aliskiren as used herein encompasses a pharmacologically acceptable salt thereof.
Vasodilators: Vasodilators work by dilating arterioles. However, vasodilatation by itself causes increased sympathetic outflow to the heart, leading to tachycardia and increased contraction.
Diazoxide (Brand: Proglycem 10 mg, 20 mg, 30 mg, 40 mg, and 50 mg) is 7-chloro-3-methyl-2H-1,2,4-benzothiadiazine 1,1-dioxide and diazoxide as used herein encompasses a pharmacologically acceptable salt thereof.
Hydralazine (Brand: Apresoline, Dralzine 10 mg, 25 mg, 50 mg, and 100 mg tablets) is 1-hydrazinophthalazine monohydrochloride and hydralazine as used herein encompasses a pharmacologically acceptable salt thereof.
Minoxidil (Brand: Loniten 2.5 mg or 10 mg tablets) is 2,4-pyrimidinediamine,6-(1-piperidinyl)-, 3-oxide (mw=209.25) and minoxidil used herein encompasses a pharmacologically acceptable salt thereof.
Nitroprusside (Brand Nipride, Nitropress, Sodium Nitroprusside 10 mg and 50 mg injection) is disodium pentacyanonitrosylferrate(2-) dihydrate and nitroprussidejk as used herein encompasses a pharmacologically acceptable salt thereof.
Potassium Channel activators: potassium-channel activators enhance the movement of potassium ions through the protein channels in cell membranes, reducing the sensitivity of smooth muscle cells in the walls of arteries to the normal stimuli to contract. The result is widening of the arteries. Potassium-channel activators may be used for improving the blood supply to the heart muscle in angina pectoris.
Embodiments include: prikalim, bimakalim, cromakalim, emakalim, nicorandil, and pinacidil.

Further, in the case where the above compounds from Lists 1, 2A, 2B, or 2C have an asymmetric carbon, the present disclosure also encompasses optical isomers and mixtures of isomers thereof. Further, the present disclosure also encompasses hydrates of the compounds.

### Formulation:

The pharmaceutical compositions according to the present disclosure may be prepared in a manner known in the art and are those suitable for suppositories, enteral and parenteral administration to mammals, including man, comprising a therapeutically effective amount of the one or more pharmacologically active compounds, alone or in combination, along with one or more pharmaceutically acceptable carriers.

### Example #1

TABLE-001
Tablet Composition Alprazolam granulation:

| No | Item | Mg/dose | % |
|---|---|---|---|
| 1 | Alprazolam | 0.25 | 0.5 |
| 2 | Hypromellose USP (K 100M) | 4.00 | 8.0 |
| 3 | Povidone USP | 0.50 | 1.0 |
| 4 | Colloidal silicon dioxide NF | 0.20 | 0.4 |
| 5 | Lactose monohydrate NF | 23.65 | 47.3 |
| 6 | Microcrystalline cellulose USP | 20.00 | 40.0 |
| | (Avicel PH 101) | | |
| 7 | Talc USP | 0.80 | 1.6 |
| 8 | Magnesium Stearate USP | 0.60 | 1.2 |
| 9 | Purified water | N/A | |

TABLE-002
Tablet Composition Nebivolol granulation:

| No | Item | Mg/dose | % |
|---|---|---|---|
| 1 | Nebivolol Hydrochloride | 2.500 | 4.20 |
| 2 | Polysobrate 80 USP | 0.750 | 1.30 |
| 3 | Hypromellose USP (E5) | 1.000 | 1.70 |
| 4 | Lactose Monohydrate NF | 25.000 | 41.70 |
| 5 | Microcrystalline cellulose USP | 29.260 | 48.80 |
| 6 | Colloidal silicon dioxide NF | 0.500 | 0.80 |
| 7 | Sodium lauryl sulphate USP | 0.500 | 0.80 |
| 8 | Magnesium Stearate USP | 0.500 | 0.80 |
| 9 | Purified water | N/A | N/A |

### Method of Manufacture:

Alprazolam Granules:

a) Mix Item #9 with Item #3 and dissolve until clear solution is obtained. This is the binder solution.
b) Pass Item #1, 2, 4, 5 and 6 thru a #18 mesh. Mix for 2 minutes in the High Shear granulator.
c) Spray binder solution onto the blend from step b and granulate for 5 min.
d) Dry the material from step c)
e) Pass dried granules from step d) thru a #20 mesh.
f) The dried granules from step e) can optionally be milled thru a Comill.
g) Blend screened granules of step f) with Items #7 & 8 (prescreen excipients thru a #18 mesh) and Blend in a v-blender for 3 minutes.
h) Compress the blend from step g).

Nebivolol Granules:

a) Mix Item #9 with Item #2 & 3 and dissolve, till clear solution is obtained. This is the binder solution.
b) Pass Item #1, 4, 5 and 6 thru a #18 mesh. Mix for 2 minutes in the High Shear granulator.
c) Spray binder solution onto the blend from step b and granulate for 5 min.
d) Dry the material from step c)
e) Pass dried granules from step d) thru a #20 mesh.
f) The dried granules from step e) can optionally be milled thru a Comill.
g) Blend screened granules of step f) with Items #7 & 8 (prescreen excipients thru a #20 mesh) and Blend in a v-blender for 3 minutes.
h) Compress the blend from step g).

In one aspect, the Alprazolam granules is compressed into 0.25 mg tablet, 0.5 mg tablets, 1 mg tablets, and 2 mg tablets as individual tablets and nebivolol granules is compressed into 2.5 mg 5 mg, 10 mg and 20 mg tablets.

In one aspect the Alprazolam granules and Nebivolol granules was compressed into matrix or bilayered tablets in the following combination:

Alprazolam 0.25 mg/2.5 mg Nebivolol tablet, Alprazolam 0.25 mg/5 mg Nebivolol tablet,
Alprazolam 0.25 mg/10 mg, Alprazolam 0.25 mg/20 mg Nebivolol tablet.
Alprazolam 0.5 mg/2.5 mg Nebivolol tablet, Alprazolam 0.5 mg/5 mg Nebivolol tablet,
Alprazolam 0.5 mg/10 mg, Alprazolam 0.5 mg/20 mg Nebivolol tablet.
Alprazolam 1.0 mg/2.5 mg Nebivolol tablet, Alprazolam 1.0 mg/5 mg Nebivolol tablet,
Alprazolam 1.0 mg/10 mg tablet, Alprazolam 1.0 mg/20 mg Nebivolol tablet
Alprazolam 2.0 mg/2.5 mg Nebivolol tablet, Alprazolam 2.0 mg/5 mg Nebivolol tablet,
Alprazolam 2.0 mg/10 mg tablet, Alprazolam 2.0 mg/20 mg Nebivolol tablet

In one aspect, the Alprazolam granules was compressed into tablets and Nebivolol granules will remain as granules, a capsule will be filled containing tablets of Alprazolam and granules of Nebivolol, in the following combination:

Alprazolam 0.25 mg/2.5 mg Nebivolol capsules, Alprazolam 0.25 mg/5 mg Nebivolol capsules,
Alprazolam 0.25 mg/10 mg, Alprazolam 0.25 mg/20 mg Nebivolol capsules.
Alprazolam 0.5 mg/2.5 mg Nebivolol capsules, Alprazolam 0.5 mg/5 mg Nebivolol capsules,
Alprazolam 0.5 mg/10 mg capsules, Alprazolam 0.5 mg/20 mg Nebivolol capsules.
Alprazolam 1.0 mg/2.5 mg Nebivolol capsules, Alprazolam 1.0 mg/5 mg Nebivolol capsules,
Alprazolam 1.0 mg/10 mg capsules, Alprazolam 1.0 mg/20 mg Nebivolol capsules.
Alprazolam 2.0 mg/2.5 mg Nebivolol capsules, Alprazolam 2.0 mg/5 mg Nebivolol capsules,
Alprazolam 2.0 mg/10 mg capsules, Alprazolam 2.0 mg/20 mg Nebivolol capsules.

In one aspect, the Alprazolam granules will remain as granules and Nebivolol granules will remain as granules, and these granules can be blended together or filled as one after another into a capsule, as in the following combination:

Alprazolam 0.25 mg/2.5 mg Nebivolol capsules, Alprazolam 0.25 mg/5 mg Nebivolol capsules,
Alprazolam 0.25 mg/10 mg, Alprazolam 0.25 mg/20 mg Nebivolol capsules.
Alprazolam 0.5 mg/2.5 mg Nebivolol capsules, Alprazolam 0.5 mg/5 mg Nebivolol capsules,
Alprazolam 0.5 mg/10 mg capsules, Alprazolam 0.5 mg/20 mg Nebivolol capsules.
Alprazolam 1.0 mg/2.5 mg Nebivolol capsules, Alprazolam 1.0 mg/5 mg Nebivolol capsules,
Alprazolam 1.0 mg/10 mg capsules, Alprazolam 1.0 mg/20 mg Nebivolol capsules.
Alprazolam 2.0 mg/2.5 mg Nebivolol capsules, Alprazolam 2.0 mg/5 mg Nebivolol capsules,
Alprazolam 2.0 mg/10 mg capsules, Alprazolam 2.0 mg/20 mg Nebivolol capsules.

### Example #2

TABLE-003
Tablet Composition Alprazolam granulation:

| No | Item | Mg/dose | % |
|---|---|---|---|
| 1 | Alprazolam | 0.125 | 0.25 |
| 2 | Hypromellose USP (K 100M) | 4.000 | 8.00 |
| 3 | Povidone USP | 0.500 | 1.00 |
| 4 | Colloidal silicon dioxide NF | 0.200 | 0.40 |
| 5 | Lactose monohydrate NF | 23.775 | 47.55 |
| 6 | Microcrystalline cellulose USP | 20.000 | 40.00 |
| | (Avicel PH 101) | | |
| 7 | Talc USP | 0.800 | 1.60 |
| 8 | Magnesium Stearate USP | 0.600 | 1.20 |
| 9 | Purified water | N/A | |

TABLE-004
Tablet Composition Nebivolol granulation:

| No | Item | Mg/dose | % |
|---|---|---|---|
| 1 | Nebivolol Hydrochloride | 2.500 | 4.20 |
| 2 | Polysobrate 80 USP | 0.750 | 1.30 |
| 3 | Hypromellose USP (E5) | 1.000 | 1.70 |
| 4 | Lactose Monohydrate NF | 25.000 | 41.70 |
| 5 | Microcrystalline cellulose USP | 29.260 | 48.80 |
| 6 | Colloidal silicon dioxide NF | 0.500 | 0.80 |
| 7 | Sodium lauryl sulphate USP | 0.500 | 0.80 |
| 8 | Magnesium Stearate USP | 0.500 | 0.80 |
| 9 | Purified water | N/A | N/A |

### Method of Manufacture

Alprazolam Granules:

a) Mix Item #9 with Item #3 and dissolve, until clear solution is obtained. This is the binder solution.
b) Pass Item #1, 2, 4, 5 and 6 thru a #18 mesh. Mix for 2 minutes in the High Shear granulator.
c) Spray binder solution onto the blend from step b and granulate for 5 min.
d) Dry the material from step c)
e) Pass dried granules from step d) thru a #20 mesh.
f) The dried granules from step e) can optionally be milled thru a Comill.
g) Blend screened granules of step f) with Items #7 &8 (prescreen excipients thru a #18 mesh) and Blend in a v-blender for 3 minutes. h) Compress the blend from step g).

Nebivolol Granules:

a) Mix Item #9 with Item #2 & 3 and dissolve, until clear solution is obtained. This is the binder solution.
b) Pass Item #1, 4, 5, and 6 thru a #18 mesh. Mix for 2 minutes in the High Shear granulator.
c) Spray binder solution onto the blend from step b and granulate for 5 min.
d) Dry the material from step c)
e) Pass dried granules from step d) thru a #20 mesh.
f) The dried granules from step e) can optionally be milled thru a Comill.
g) Blend screened granules of step f) with Items #7 & 8 (prescreen excipients thru a #20 mesh) and Blend in a v-blender for 3 minutes.
h) Compress the blend from step g).

In some aspects, the Alprazolam granules is compressed into 0.125 mg tablet, 0.25 mg tablets, 0.5 mg tablets and 1.0 mg tablets as individual tablets and nebivolol granules is compressed into 2.5 mg 5 mg, 10 mg and 20 mg tablets.

In some aspect the Alprazolam granules and Nebivolol granules was compressed into matrix or bilayered tablets in the following combination:

Alprazolam 0.125 mg/2.5 mg Nebivolol tablet, Alprazolam 0.125 mg/5 mg Nebivolol tablet,
Alprazolam 0.125 mg/10 mg, Alprazolam 0.125 mg/20 mg Nebivolol tablet.
Alprazolam 0.25 mg/2.5 mg Nebivolol tablet, Alprazolam 0.25 mg/5 mg Nebivolol tablet,
Alprazolam 0.25 mg/10 mg, Alprazolam 0.25 mg/20 mg Nebivolol tablet.
Alprazolam 0.5 mg/2.5 mg Nebivolol tablet, Alprazolam 0.5 mg/5 mg Nebivolol tablet,
Alprazolam 0.5 mg/10 mg tablet, Alprazolam 0.5 mg/20 mg Nebivolol tablet
Alprazolam 1.0 mg/2.5 mg Nebivolol tablet, Alprazolam 1.0 mg/5 mg Nebivolol tablet,
Alprazolam 1.0 mg/10 mg tablet, Alprazolam 1.0 mg/20 mg Nebivolol tablet

In some aspects, the Alprazolam granules was compressed into tablets and Nebivolol granules will remain as granules, a capsule will be filled containing tablets of Alprazolam and granules of Nebivolol, in the following combination:

Alprazolam 0.125 mg/2.5 mg Nebivolol capsules, Alprazolam 0.125 mg/5 mg Nebivolol capsules, Alprazolam 0.125 mg/10 mg, Alprazolam 0.125 mg/20 mg Nebivolol capsules.
Alprazolam 0.25 mg/2.5 mg Nebivolol capsules, Alprazolam 0.25 mg/5 mg Nebivolol capsules,
Alprazolam 0.25 mg/10 mg capsules, Alprazolam 0.25 mg/20 mg Nebivolol capsules.
Alprazolam 0.5 mg/2.5 mg Nebivolol capsules, Alprazolam 0.5 mg/5 mg Nebivolol capsules,
Alprazolam 0.5 mg/10 mg capsules, Alprazolam 0.5 mg/20 mg Nebivolol capsules.
Alprazolam 1.0 mg/2.5 mg Nebivolol capsules, Alprazolam 1.0 mg/5 mg Nebivolol capsules,
Alprazolam 1.0 mg/10 mg capsules, Alprazolam 1.0 mg/20 mg Nebivolol capsules

In some aspects, the Alprazolam granules will remain as granules and Nebivolol granules will remain as granules, and these granules can be blended together or filled as one after another into a capsule, as in the following combination:

Alprazolam 0.125 mg/2.5 mg Nebivolol capsules, Alprazolam 0.125 mg/5 mg Nebivolol capsules, Alprazolam 0.125 mg/10 mg, Alprazolam 0.125 mg/20 mg Nebivolol capsules.
Alprazolam 0.25 mg/2.5 mg Nebivolol capsules, Alprazolam 0.25 mg/5 mg Nebivolol capsules,
Alprazolam 0.25 mg/10 mg capsules, Alprazolam 0.25 mg/20 mg Nebivolol capsules.
Alprazolam 0.5 mg/2.5 mg Nebivolol capsules, Alprazolam 0.5 mg/5 mg Nebivolol capsules,
Alprazolam 0.5 mg/10 mg capsules, Alprazolam 0.5 mg/20 mg Nebivolol capsules.
Alprazolam 1.0 mg/2.5 mg Nebivolol capsules, Alprazolam 1.0 mg/5 mg Nebivolol capsules,
Alprazolam 1.0 mg/10 mg capsules, Alprazolam 1.0 mg/20 mg Nebivolol capsules.

### Example #3

TABLE-005
Tablet Composition Triazolam granulation:

| No | Item | Mg/dose | % |
|---|---|---|---|
| 1 | Triazolam | 0.25 | 0.5 |
| 2 | Hydroxy Propyl Methylcellulose | 4.00 | 8.0 |
| | USP (K 100M) | | |
| 3 | Povidone USP | 0.50 | 1.0 |
| 4 | Colloidal silicon dioxide NF | 0.20 | 0.4 |
| 5 | Lactose monohydrate NF | 23.65 | 47.3 |
| 6 | Microcrystalline cellulose USP | 20.00 | 40.0 |
| | (Avicel PH 101) | | |
| 7 | Talc USP | 0.80 | 1.6 |
| 8 | Magnesium Stearate USP | 0.60 | 1.2 |
| 9 | Purified water | N/A | |

TABLE-006
Tablet Composition Nebivolol granulation:

| No | Item | Mg/dose | % |
|---|---|---|---|
| 1 | Nebivolol Hydrochloride | 2.500 | 4.20 |
| 2 | Polysobrate 80 USP | 0.750 | 1.30 |
| 3 | Hypromellose USP (E5) | 1.000 | 1.70 |
| 4 | Lactose Monohydrate NF | 25.000 | 41.70 |
| 5 | Microcrystalline cellulose USP | 29.260 | 48.80 |
| 6 | Colloidal silicon dioxide NF | 0.500 | 0.80 |
| 7 | Sodium lauryl sulphate USP | 0.500 | 0.80 |
| 8 | Magnesium Stearate USP | 0.500 | 0.80 |
| 9 | Purified water | N/A | N/A |

### Method of Manufacture

Triazolam Granules:

a) Mix Item #9 with Item #3 and dissolve, until clear solution is obtained. This is the binder solution.
b) Pass Item #1, 2, 4, 5 and 6 thru a #18 mesh. Mix for 2 minutes in the High Shear granulator.
c) Spray binder solution onto the blend from step b and granulate for 5 min.
d) Dry the material from step c)
e) Pass dried granules from step d) thru a #20 mesh.
f) The dried granules from step e) can optionally be milled thru a Comill.
g) Blend screened granules of step f) with Items #7 & 8 (prescreen excipients thru a #18 mesh) and Blend in a v-blender for 3 minutes.
h) Compress the blend from step g).

Nebivolol Granules:

a) Mix Item #9 with Item #2 & 3 and dissolve, until clear solution is obtained. This is the binder solution.
b) Pass Item #1, 4, 5 and 6 thru a #18 mesh. Mix for 2 minutes in the High Shear granulator.
c) Spray binder solution onto the blend from step b and granulate for 5 min.
d) Dry the material from step c)
e) Pass dried granules from step d) thru a #20 mesh.
f) The dried granules from step e) can optionally be milled thru a Comill.
g) Blend screened granules of step f) with Items #7 & 8 (prescreen excipients thru a #20 mesh) and Blend in a v-blender for 3 minutes.
h) Compress the blend from step g).

In some aspects, the triazolam granules is compressed into 0.25 mg tablets as individual tablets and nebivolol granules is compressed into 2.5 mg 5 mg, 10 mg and 20 mg tablets.

In some aspects, the triazolam granules and nebivolol granules was compressed into matrix or bilayered tablets in the following combination:

Triazolam 0.25 mg/2.5 mg Nebivolol tablet, Triazolam 0.25 mg/5 mg Nebivolol tablet,
Triazolam 0.25 mg/10 mg, Triazolam 0.25 mg/20 mg Nebivolol tablet.

In some aspects, the triazolam granules was compressed into tablets and nebivolol granules will remain as granules, a capsules will be filled containing tablets of Triazolam and granules of Nebivolol, in the following combination:

Triazolam 0.25 mg/2.5 mg Nebivolol capsules, Triazolam 0.25 mg/5 mg Nebivolol capsules,
Triazolam 0.25 mg/10 mg, Triazolam 0.25 mg/20 mg Nebivolol capsules.

In some aspects, the triazolam granules will remain as granules and nebivolol granules will remain as granules, and these granules can be blended together or filled as one after another into a capsule, as in the following combination:

Triazolam 0.25 mg/2.5 mg Nebivolol capsules, Triazolam 0.25 mg/5 mg Nebivolol capsules,
Triazolam 0.25 mg/10 mg, Triazolam 0.25 mg/20 mg Nebivolol capsules.

In some aspects, the above combination used to treat or reduce morning hypertension and nocturnal hypertension. As will be apparent, the use will depend upon the time of administration, preferably for morning hypertension the combination will be administered in the morning and preferably for nocturnal hypertension, when administered during nighttime.

In the present disclosure, preferred benzodiazepines are those agents that have been marketed, such as, e.g. alprazolam and clonazepam, as well as for betablockers employed in the present disclosure, of which nebivolol and atenolol are preferred. The most preferred diuretic is hydrochlorothiazide (HCTZ).

Also provided herein in another aspect is a method of treating morning hypertension or nocturnal hypertension in a subject in need thereof comprising administering the pharmaceutical composition comprising. (i) one or more benzodiazepines; and (ii) one or more antihypertensive agents, such as calcium channel blocker or a beta-blocker, wherein the dose of each component (i) is 0.01% to 50% of the component (ii) agent(s).

In some aspects, the treatment results in a reduction of systolic blood pressure (SBP) of about 10 mmHg or greater in morning or nocturnal hypertension. In some aspects, the treatment results in a reduction of diastolic blood pressure (DBP) of about 5 mmHg or greater in morning or nocturnal hypertension. In some aspects, the treatment is the initial or first-line treatment of morning hypertension or nocturnal hypertension.

The present disclosure recognizes the technical effects of low-dose combination therapy set forth herein for benzodiazepines, including but not limited to, the use of low-doses to avoid or ameliorate side effects while retaining or improving benefits, the synergistic therapeutic benefits of certain drug combinations, the early introduction of combination therapy to improve therapeutic effects. Described herein are low-dose combination compositions for the treatment of morning hypertension or nocturnal hypertension, including the initial or first-line treatment of morning or nocturnal hypertension.

In some aspects, the dose of the benzodiazepines is about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, r about 85%, about 86%, about 87%, about 88%, about 89%, or about 90% of the lowest hypnotic, sedative, or anxiolytic therapeutic dose.

In the disclosure, the phrase administered “simultaneously” is not particularly limited as long as it is an administration form capable of performing administration at substantially the same time, however, it is preferred to perform administration as a single composition or as kit form.

In the disclosure, the phrase administered “separately at a time interval” is not particularly limited as long as it is an administration form capable of performing administration separately at different times, however, for example, it refers to that first, a benzodiazepine is administered, and then, after a predetermined time, a calcium antagonist is administered, or first, a calcium antagonist or a diuretic is administered, and then, after a predetermined time, an benzodiazepine is administered in the same manner as described above.

One embodiment of the present disclosure includes a pharmaceutical composition for administering simultaneously or separately at a time interval the benzodiazepine and the angiotensin II receptor antagonist, the calcium antagonist, or the diuretic of the present disclosure. One embodiment may further lower the blood pressure during morning hours or at nocturnal hours by administering (i) the anxiolytics, sedatives, or hypnotics, such as alprazolam, clonazepam, lorazepam, melatonin, and non-benzodiazepine sedatives like zolipderm, zaleplon, eszopiclone, orexin receptor antagonist, and (ii) one or more medicine to treat high blood pressure to act as described in this description.

Based on the activity described above, the pharmaceutical composition of the present disclosure can be used for the treatment of morning/nocturnal hypertension, a heart disease, including but not limited to angina pectoris, myocardial infarction, arrhythmia, including sudden death, heart failure, or cardiac hypertrophy, a kidney disease, such as diabetic nephropathy, glomerulonephritis, or nephrosclerosis, a cerebrovascular disease, such as cerebral infarction or intracerebral hemorrhage, or a vascular disorder, such as arteriosclerosis, restenosis after PTCA, or peripheral circulatory disturbance. Incidentally, by using the MR antagonist and the angiotensin II receptor antagonist, the calcium antagonist, or the diuretic of the present disclosure in combination, an improved effect is exhibited as compared with the case where each agent is administered singly.

Benzodiazepines and the beta blockers, angiotensin II receptor antagonist, the calcium antagonist, or the diuretic serving as the active ingredients of the pharmaceutical composition of the present disclosure can be prepared in the form of separate unit dosage forms each containing a single agent alone, or can be prepared physically in the form of one unit dosage form by mixing these agents.

In the case where the pharmaceutical composition of the present disclosure is used as a therapeutic agent for any of the above-mentioned diseases, the benzodiazepines and the beta blockers, the angiotensin II receptor antagonist, the calcium antagonist, or the diuretic serving as the active ingredients of the pharmaceutical composition of the present disclosure can be administered orally in the form of a tablet, a capsule, a granule, a powder, a syrup, sublingual spray or a nasal spray or the like, or parenterally in the form of an injection, a suppository, or the like, which is produced according to a known method using the respective agents by themselves, or by using also a suitable pharmacologically acceptable additive such as an excipient, rate controlling polymer, a lubricant, a binder, a disintegrant, an emulsifier, a stabilizer, a corrigent, or a diluent. Incidentally, the all the above pharmaceutical components described above are agents to be generally administered orally, and therefore, the pharmaceutical of the present disclosure is desirably administered orally.

In some aspects, the benzodiazepines can administered alone or in combination with cholesterol lowering drugs like Simvastatin, Aspirin, blood thinners like Apaxiban, ischemic treatment drugs like Ranolazine, and other drugs that are useful for adjunct treatment of cardiovascular diseases like (acute and chronic) congestive heart failure, left ventricular dysfunction and hypertrophic cardiomyopathy, myocardial infarction and its sequelae, supraventricular and ventricular arrhythmias, atrial fibrillation or atrial flutter, atherosclerosis, angina (whether stable or unstable), renal insufficiency (diabetic and non-diabetic), heart failure, angina pectoris, diabetes, secondary aldosteronism, primary and secondary pulmonary hyperaldosteronism, primary and pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, Raynaud's disease, luminal hyperplasia, and cognitive dysfunction (such as Alzheimer's).

### In Vivo Testing

Test of Combined Administration of Benzodiazepine and Antihypertensive Agents to Evaluate the Treatment Action on Morning Hypertension

Hypertensive elderly patients (n=6) at age group of 60 years and older, non smokers were allowed to take their regular antihypertensive medications and tested for the blood pressure during the night before bed time and within 2 hours after awakening and every 3 hours during the day time for a two week period for initial evaluation, followed by two week administration period with a combination with a benzodiazepine and nebivolol. Morning BP surge (MBPS) is defined as the sleep-trough surge, calculated by subtracting the morning BP (mean of three readings over 2 h just after waking up) from the lowest nocturnal BP before going to bed (mean of three readings around the lowest nighttime)

Example for a Patient Taking Regular Medication:

Morning; Ranolazine 500 mg tablets, Amlodpine 5 mg
Night time: Ranolazine 500 mg, Asprin 75 mg, Atrovastatin 20 mg, Clopidogrel 75 mg and Nebivolol 5 mg

TABLE-007
initial parameters of morning hypertension

| Variable | Total Patient (n = 6) |
|---|---|
| Age (Mean +/− SD) | 67.9 +/− 7.4 |
| 24-h mean SBP (mmHg) | 141.4 ± 11.8 |
| Daytime mean SBP (mmHg) | 145.1 ± 12.9 |
| Nighttime mean SBP (mmHg) | 132.7 ± 10.1 |
| MBPS (mmHg) | 24.2 ± 9.1 |

Treatment with Benzodiazepine and Nebivolol

Alprazolam tablets were added to the regimen for the above same patients along with their routine medication for hypertension. Blood pressure were measured and the results are as follow:

Example for a Patient Taking Regular Medication:

Morning; Ranolazine 500 mg tablets, Amlodpine 5 mg
Night time: Ranolazine 500 mg, Asprin 75 mg, Atrovastatin 20 mg, Clopidogrel 75 mg, Nebivolol 5 mg and Alprazolam 2.5 mg

TABLE-008
Data after Treatment with Benzodiazepine and Nebivolol

| Variable | Total Patient (n = 6) |
|---|---|
| 24-h mean SBP (mmHg) | 135.4 ± 12.8 |
| Daytime mean SBP (mmHg) | 138.8 ± 10.3 |
| Nighttime mean SBP (mmHg) | 133..6 ± 11.4 |
| MBPS (mmHg) | 9.6 ± 6.9 |

With the combined administration of the benzodiazepines at nighttime along with the antihypertensive drugs, an improved morning pressure lowering was observed. A reduction of morning blood pressure elevation or morning hypertension was confirmed.

According to the present disclosure, a pharmaceutical combination for the treatment of morning hypertension or nocturnal hypertension or a disease derived from hypertension is obtained. More specifically, a pharmaceutical combination for the treatment of morning/nocturnal hypertension, a heart disease, such as angina pectoris, myocardial infarction, stroke, arrhythmia (including sudden death), heart failure, Marton syndrome or cardiac hypertrophy, left ventricular hypertrophy, a kidney disease, such as diabetic nephropathy, glomerulonephritis, or nephrosclerosis, atherosclerotic plaque phenotype, a cerebrovascular disease, such as cerebral infarction or intracerebral hemorrhage, or a vascular disorder, such as arteriosclerosis, restenosis after PTCA, or peripheral circulatory disturbance is herein described.

Enumerated embodiments of the present disclosure include:

1. A method for controlling either morning or nocturnal hypertension to a subject in need thereof comprising administering:
a) a therapeutically effective amount of one or more of an anxiolytic, sedative, and hypnotic agent; and
b) a therapeutically effective amount of one or more anti-hypertensive agent.
   2. The method of embodiment 1, wherein the anxiolytic, sedative and hypnotic agent is selected from the group consisting of: a benzodiazepine, alprazolam, clonazepam, lorazepam, melatonin, a non-benzodiazepine, zolipderm, zaleplon, eszopiclone, and an orexin receptor antagonist.
   3. The method of embodiment 2, wherein the anxiolytic, sedative and hypnotic agent is a benzopiazepine.
   4. The method of any one of embodiments 1 to 3, wherein the antihypertensive agent is selected from the group consisting of a diuretic, chlorothizide, furosemide, spironolactone, indapamide, a beta-blockers, atenolol, metoprolol tartrate, bisoprolol fumarate, propanolol hydrochloride, an ACE inhibitor, enalapril, moexipril, Lisinopril, captopril, and angiotensin II receptor blocker, valsartan, telmisartan, irbesartan, a calcium channel blocker, amlodipine besylate, diltiazem hydrochloride, nifedipine, an alpha blocker, doxazosin mesylate, prazosin hydrochloride, terazosin hydrochloride, an alpha-2 receptor agonist, methyldopa, a combined alpha and beta blocker, carvediol, labetalol hydrochloride, a central agonist, clonidine hydrochloride, guanfacine hydrochloride, a peripheral adrenergic inhibitor, guanadrel, reserpine, a vasodilator, minoxidil, and hydralazine hydrochloride.
   5. The method of embodiment 4, wherein the any-hypertensive is selected from a beta blocker, a calcium channel blocker, a diuretic, or an ACE inhibitor.
   6. The method of any one of embodiments 1 to 5, wherein the agents are administered as a fixed dose combination.
   7. The method of any one of embodiments 1 to 6, wherein the anti-hypertensive is provided as
(i) nebivolol administered in an amount from about 1 mg to about 60 mg;
(ii) metoprolol administered in an amount from about 10 mg to about 600 mg
(iii) amlodipine administered in an amount from about 1 mg to about 60 mg;
(iv) valsartan administered in an amount from about 10 mg to about 650 mg; or
(v) hydrochlorothiazide administered in an amount from about 5 mg to about 200 mg.
   8. The method of any one of embodiments 1 to 7, wherein the sedative is alprazolam administered in about 0.01 mg to about 10 mg.
   9. The method of any one of embodiments 1 to 8, wherein the therapeutically effective amount of an agent for the present method is less than a standard therapeutic dosage of the agent.
   10. The method of any one of embodiments 1 to 9, wherein the therapeutically effective amount of an agent for the present method is about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90% of the lowest therapeutic dose.
   11. A kit comprising:
a. a therapeutically effective amount in unit dose form of one or more of an anxiolytic, sedative, and hypnotic agent;
b. a therapeutically effective amount in unit dose form of one or more anti-hypertensive agent; and
c. instructions for the administration of the agents.
   12. The kit of embodiment 11, wherein the anxiolytic, sedative and hypnotic agent is selected from the group consisting of: a benzodiazepine, alprazolam, clonazepam, lorazepam, melatonin, a non-benzodiazepine, zolipderm, zaleplon, eszopiclone, and an orexin receptor antagonist.
   13. The kit of embodiment 12, wherein the anxiolytic, sedative and hypnotic agent is a benzodiazepine.
   14. The kit of any one of embodiments 11 to 13, wherein the antihypertensive agent is selected from the group consisting of a diuretic, chlorothizide, furosemide, spironolactone, indapamide, a beta-blockers, atenolol, metoprolol tartrate, bisoprolol fumarate, propanolol hydrochloride, an ACE inhibitor, enalapril, moexipril, Lisinopril, captopril, and angiotensin II receptor blocker, valsartan, telmisartan, irbesartan, a calcium channel blocker, amlodipine besylate, diltiazem hydrochloride, nifedipine, an alpha blocker, doxazosin mesylate, prazosin hydrochloride, terazosin hydrochloride, an alpha-2 receptor agonist, methyldopa, a combined alpha and beta blocker, carvediol, labetalol hydrochloride, a central agonist, clonidine hydrochloride, guanfacine hydrochloride, a peripheral adrenergic inhibitor, guanadrel, reserpine, a vasodilator, minoxidil, and hydralazine hydrochloride.
   15. The kit of embodiment 14, wherein the anti-hypertensive is selected from a beta blocker, a calcium channel blocker, a diuretic, or an ACE inhibitor.
   16. The kit of any one of embodiments 11 to 15, wherein the agents' unit dose forms are provided as a single, fixed dose combination.
   17. The kit of any one of embodiments 11 to 16, a benzodiazepine and anti-hypertensive agents, wherein the anti-hypertensive is provided as
a) nebivolol in an amount from about 1 mg to about 60 mg;
b) metoprolol in an amount from about 10 mg to about 600 mg
c) amlodipine in an amount from about 1 mg to about 60 mg;
d) valsartan in an amount from about 10 mg to about 650 mg; or
e) hydrochlorothiazide in an amount from about 5 mg to about 200 mg.
   18. The kit of any one of embodiments 11 to 17, wherein the sedative is alprazolam in an amount of about 0.01 mg to about 10 mg.
   19. The kit of any one of embodiments 11 to 18, wherein the therapeutically effective amount of an agent for the present method is less than a standard therapeutic dosage of the agent.
   20. The kit of any one of embodiments 11 to 19, wherein the therapeutically effective amount of an agent for the present method is about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90% of the lowest therapeutic dose.
   21. The kit of any one of embodiments 11 to 20, further comprising a therapeutically effective amount in unit dose form of isosorbide mono- or di-nitrate.
   22. The kit of any one of embodiments 11 to 20, wherein the instructions provide guidance on the use of the dose units for:
a. the treatment of a condition or disease selected from the group consisting of: morning hypertension, hypertension, nocturnal hypertension, stroke, peripheral edema, heart failure, congestive heart failure, left ventricular dysfunction, hypertrophic cardiomyopathy, diabetic cardiac myopathy, supraventricular arrhythmia, ventricular arrhythmia, atrial fibrillation, atrial flutter, detrimental vascular remodeling, myocardial infarction, sequelae of myocardial infarction, atherosclerosis, angina, renal insufficiency, angina pectoris, diabetes, secondary aldosteronism, pulmonary hypertension, and renal failure; or
b. the management of a condition or disease selected from the group consisting of: morning hypertension, nocturnal hypertension, peripheral vascular disease, Raynaud's disease, luminal hyperplasia, cognitive dysfunction, glaucoma, and stroke.
   23. A pharmaceutical combination composition comprising:
a. a therapeutically effective amount of one or more of an anxiolytic, sedative, and hypnotic agent;
b. a therapeutically effective amount of one or more anti-hypertensive agent; and
c. one or more pharmaceutically acceptable carrier.
   24. The composition of embodiment 23, wherein each agent individually is provided as an immediate release, or as a delayed, controlled, or extended release formulation.
   25. The composition of embodiment 23, wherein both agents are provided as an immediate release, or as a delayed, controlled, or extended release formulation.
   26. The composition of any one of embodiments 23 to 25, wherein the composition is formulated for a subject in need of treating hypertension associated with a definable time period.
   27. The composition of embodiment 26, wherein the time period is morning.
   28. The composition of embodiment 26, wherein the time period is night.
   29. The composition of any one of embodiments 23 to 28, wherein the anxiolytic, sedative and hypnotic agent is selected from the group consisting of: a benzodiazepine, alprazolam, clonazepam, lorazepam, melatonin, a non-benzodiazepine, zolipderm, zaleplon, eszopiclone, and an orexin receptor antagonist.
   30. The composition of embodiment 29, wherein the anxiolytic, sedative and hypnotic agent is a benzodiazepine.
   31. The composition of any one of embodiments 23 to 30, wherein the antihypertensive agent is selected from the group consisting of a diuretic, chlorothizide, furosemide, spironolactone, indapamide, a beta-blockers, atenolol, metoprolol tartrate, bisoprolol fumarate, propanolol hydrochloride, an ACE inhibitor, enalapril, moexipril, Lisinopril, captopril, and angiotensin II receptor blocker, valsartan, telmisartan, irbesartan, a calcium channel blocker, amlodipine besylate, diltiazem hydrochloride, nifedipine, an alpha blocker, doxazosin mesylate, prazosin hydrochloride, terazosin hydrochloride, an alpha-2 receptor agonist, methyldopa, a combined alpha and beta blocker, carvediol, labetalol hydrochloride, a central agonist, clonidine hydrochloride, guanfacine hydrochloride, a peripheral adrenergic inhibitor, guanadrel, reserpine, a vasodilator, minoxidil, and hydralazine hydrochloride.
   32. The composition of embodiment 31, wherein the any-hypertensive is selected from a beta blocker, a calcium channel blocker, a diuretic, or an ACE inhibitor.
   33. The composition of any one of embodiments 23 to 32, wherein the agents are formulated as a fixed dose combination.
   34. The composition of any one of embodiments 23 to 33, wherein the anti-hypertensive is provided as:
(i) nebivolol in an amount from about 1 mg to about 60 mg;
(ii) metoprolol in an amount from about 10 mg to about 600 mg;
(iii) amlodipine in an amount from about 1 mg to about 60 mg;
(iv) valsartan in an amount from about 10 mg to about 650 mg; or
(v) hydrochlorothiazide in an amount from about 5 mg to about 200 mg.
   35. The composition of any one of embodiments 23 to 34, wherein the sedative is alprazolam in an amount of about 0.01 mg to about 10 mg.
   36. The composition of any one of embodiments 23 to 35, wherein the therapeutically effective amount of an agent for the present method is less than a standard therapeutic dosage of the agent.
   37. The composition of any one of embodiments 23 to 36, wherein the therapeutically effective amount of an agent for the present method is about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90% of the lowest therapeutic dose.
   38. The composition of any one of embodiments 23 to 37, wherein the composition is an oral solid dosage form.
   39. The composition of embodiment 38, wherein the composition is a powder, granule, dripping pill, micro-pellet, tablet, sublingual tablets, sublingual drops, bilayered tablet, matrix tablet, capsule, or lozenge.
   40. The composition of embodiment 30, wherein 90% of the benzodiazepine is released in about 30 minutes after administration.
   41. The composition of embodiment 30, wherein 90% of the benzodiazepine is released in about 60 minutes after administration.
   42. The composition of embodiment 30, wherein 90% of the benzodiazepine is released in about 4 hours after administration.
   43. The composition of embodiment 30, wherein 90% of the benzodiazepine is released in about 12 hours after administration.
   44. The composition of embodiment 30, wherein the benzodiazepine is alprazolam.
   45. The composition of embodiment 44, wherein the alprazolam is provided in an amount of about 0.10 mg, about 0.20 mg, about 0.25 mg, about 0.30 mg, about 0.40 mg, about 0.50 mg, about 0.60 mg, about 0.70 mg, about 0.80 mg, about 0.90 mg, about 1.0 mg, about 1.6 mg, about 2.0 mg, about 3.0 mg, or about 5.0 mg.
   46. The composition of embodiment 30, wherein the benzodiazepine is provided in an amount of about 0.025 mg to 40 mg.
   47. The composition of embodiment 30, wherein the benzodiazepine is formulated as a nasal spray, a sublingual spray, an oral solution, or and oral suspension.
   48. The composition of any one of embodiments 23 to 47, wherein the one or more pharmaceutically acceptable carrier is selected from the group consisting of: poly methacrylate, poly butyl methacrylate, 2-dimethylaminoethyl methacrylate, methyl methacrylate, cellulose esters, carbomers, and combinations thereof.
   49. The composition of embodiment 29, wherein the melatonin is provided in the amount of about 1.0 to about 50 mg.
   50. A method of treating either morning or nighttime hypertension for a patient in need thereof comprising administering:
a. a therapeutically effective amount of an agent that affects the sympathetic nervous system; and
b. a therapeutically effective amount of one or more anti-hypertensive agent, wherein the patient's blood pressure is reduced compared to a baseline measurement.
   51. The method of embodiment 50, wherein the patient's blood pressure is less than about 135 mm of Hg.
   52. The method of embodiment 50, wherein the patient's blood pressure is less than about 130 mm of Hg.
   53. The method of embodiment 50, wherein the patient's blood pressure is less than about 125 mm of Hg.
   54. The method of embodiment 50, wherein the patient's blood pressure is less than about 120 mm of Hg.
   55. The method of any one of embodiments 50 to 54, wherein for treatment of morning hypertension, the administration of an immediate release formulation is provided within about 1 hour of the subject's waking.
   56. The method of any one of embodiments 50 to 54, wherein for treatment of nighttime hypertension, the administration of an immediate release formulation is provided within about 1 hour of the subject's sleeping.
   57. The method of any one of embodiments 50 to 54, wherein for treatment of morning hypertension, the administration of an extended release formulation is provided within about 8 to about 12 hours of the subject's waking.
   58. The method of any one of embodiments 50 to 54, wherein for treatment of nighttime hypertension, the administration of an extended release formulation is provided within about 8 to about 12 hours of the subject's sleeping.

While the present disclosure has been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

1. A method for controlling either morning or nocturnal hypertension to a subject in need thereof comprising administering:
a) a therapeutically effective amount of one or more of an anxiolytic, sedative, and hypnotic agent; and
b) a therapeutically effective amount of one or more anti-hypertensive agent.

2. The method of claim 1, wherein the anxiolytic, sedative and hypnotic agent is selected from the group consisting of: a benzodiazepine, alprazolam, clonazepam, lorazepam, melatonin, a non-benzodiazepine, zolipderm, zaleplon, eszopiclone, and an orexin receptor antagonist.

3. (canceled)

4. The method of claim 1, wherein the antihypertensive agent is selected from the group consisting of a diuretic, chlorothizide, furosemide, spironolactone, indapamide, a beta-blockers, atenolol, metoprolol tartrate, bisoprolol fumarate, propanolol hydrochloride, an ACE inhibitor, enalapril, moexipril, Lisinopril, captopril, and angiotensin II receptor blocker, valsartan, telmisartan, irbesartan, a calcium channel blocker, amlodipine besylate, diltiazem hydrochloride, nifedipine, an alpha blocker, doxazosin mesylate, prazosin hydrochloride, terazosin hydrochloride, an alpha-2 receptor agonist, methyldopa, a combined alpha and beta blocker, carvediol, labetalol hydrochloride, a central agonist, clonidine hydrochloride, guanfacine hydrochloride, a peripheral adrenergic inhibitor, guanadrel, reserpine, a vasodilator, minoxidil, and hydralazine hydrochloride.

5. (canceled)

6. The method of claim 1, wherein the agents are administered as a fixed dose combination.

7-8. (canceled)

9. The method of claim 1, wherein the therapeutically effective amount of an agent for the present method is less than a standard therapeutic dosage of the agent.

10. (canceled)

11. A kit comprising:
a. a therapeutically effective amount in unit dose form of one or more of an anxiolytic, sedative, and hypnotic agent;
b. a therapeutically effective amount in unit dose form of one or more anti-hypertensive agent; and
c. instructions for the administration of the agents.

12. The kit of claim 11, wherein the anxiolytic, sedative and hypnotic agent is selected from the group consisting of: a benzodiazepine, alprazolam, clonazepam, lorazepam, melatonin, a non-benzodiazepine, zolipderm, zaleplon, eszopiclone, and an orexin receptor antagonist.

13. (canceled)

14. The kit of claim 11, wherein the antihypertensive agent is selected from the group consisting of a diuretic, chlorothizide, furosemide, spironolactone, indapamide, a beta-blockers, atenolol, metoprolol tartrate, bisoprolol fumarate, propanolol hydrochloride, an ACE inhibitor, enalapril, moexipril, Lisinopril, captopril, and angiotensin II receptor blocker, valsartan, telmisartan, irbesartan, a calcium channel blocker, amlodipine besylate, diltiazem hydrochloride, nifedipine, an alpha blocker, doxazosin mesylate, prazosin hydrochloride, terazosin hydrochloride, an alpha-2 receptor agonist, methyldopa, a combined alpha and beta blocker, carvediol, labetalol hydrochloride, a central agonist, clonidine hydrochloride, guanfacine hydrochloride, a peripheral adrenergic inhibitor, guanadrel, reserpine, a vasodilator, minoxidil, and hydralazine hydrochloride.

15-20. (canceled)

21. The kit of claim 11, further comprising a therapeutically effective amount in unit dose form of isosorbide mono- or di-nitrate.

22. The kit of claim 11, wherein the instructions provide guidance on the use of the dose units for:
a. the treatment of a condition or disease selected from the group consisting of: morning hypertension, hypertension, nocturnal hypertension, stroke, peripheral edema, heart failure, congestive heart failure, left ventricular dysfunction, hypertrophic cardiomyopathy, diabetic cardiac myopathy, supraventricular arrhythmia, ventricular arrhythmia, atrial fibrillation, atrial flutter, detrimental vascular remodeling, myocardial infarction, sequelae of myocardial infarction, atherosclerosis, angina, renal insufficiency, angina pectoris, diabetes, secondary aldosteronism, pulmonary hypertension, and renal failure; or
b. the management of a condition or disease selected from the group consisting of: morning hypertension, nocturnal hypertension, peripheral vascular disease, Raynaud's disease, luminal hyperplasia, cognitive dysfunction, glaucoma, and stroke.

23. A pharmaceutical combination composition comprising:
a. a therapeutically effective amount of one or more of an anxiolytic, sedative, and hypnotic agent;
b. a therapeutically effective amount of one or more anti-hypertensive agent; and
c. one or more pharmaceutically acceptable carrier.

24. The composition of claim 23, wherein either:
each agent individually is provided as an immediate release, or as a delayed, controlled, or extended release formulation; or
both agents are provided as an immediate release, or as a delayed, controlled, or extended release formulation.

25. (canceled)

26. The composition of claim 23, wherein the composition is formulated for a subject in need of treating hypertension associated with a definable time period that is selected from morning or night.

27-28. (canceled)

29. The composition of claim 23, wherein the anxiolytic, sedative and hypnotic agent is selected from the group consisting of: a benzodiazepine, alprazolam, clonazepam, lorazepam, melatonin, a non-benzodiazepine, zolipderm, zaleplon, eszopiclone, and an orexin receptor antagonist.

30. (canceled)

31. The composition of claim 23, wherein the antihypertensive agent is selected from the group consisting of a diuretic, chlorothizide, furosemide, spironolactone, indapamide, a beta-blockers, atenolol, metoprolol tartrate, bisoprolol fumarate, propanolol hydrochloride, an ACE inhibitor, enalapril, moexipril, Lisinopril, captopril, and angiotensin II receptor blocker, valsartan, telmisartan, irbesartan, a calcium channel blocker, amlodipine besylate, diltiazem hydrochloride, nifedipine, an alpha blocker, doxazosin mesylate, prazosin hydrochloride, terazosin hydrochloride, an alpha-2 receptor agonist, methyldopa, a combined alpha and beta blocker, carvediol, labetalol hydrochloride, a central agonist, clonidine hydrochloride, guanfacine hydrochloride, a peripheral adrenergic inhibitor, guanadrel, reserpine, a vasodilator, minoxidil, and hydralazine hydrochloride.

32. (canceled)

33. The composition of claim 23, wherein the agents are formulated as a fixed dose combination.

34. The composition of claim 23, wherein
the anti-hypertensive is provided as:
(i) nebivolol in an amount from about 1 mg to about 60 mg;
(ii) metoprolol in an amount from about 10 mg to about 600 mg;
(iii) amlodipine in an amount from about 1 mg to about 60 mg;
(iv) valsartan in an amount from about 10 mg to about 650 mg; or
(v) hydrochlorothiazide in an amount from about 5 mg to about 200 mg; and
the sedative is alprazolam in an amount of about 0.01 mg to about 10 mg.

35-37. (canceled)

38. The composition of claim 23, wherein the composition is an oral solid dosage form.

39-50. (canceled)

51. The method of claim 1, wherein the patient's blood pressure is reduced compared to a baseline measurement.

52. The method of claim 50, wherein the patient's blood pressure is less than about 135 mm of Hg.
